(19) United States
(12) Patent Application Publication
(10) Pub. No.: US 2006/0094039 A1
(43) Pub. Date: May 4, 2006

(54) Diagnosis of fetal aneuploidy

(76) Inventors: Ron Rosenfeld, Los Altos, CA (US); Srinivasa Nagalla, Hillsboro, OR (US)

Correspondence Address: HELLER EHRMAN LLP, 275 MIDDLEFIELD ROAD, MENLO PARK, CA, 94025-3506, US

(21) Appl. No.: 11/232,335

(22) Filed: Sep. 20, 2005

Related U.S. Application Data
(60) Provisional application No. 60/611,654, filed on Sep. 20, 2004.

(51) Int. Cl. C12Q 1/68; G01N 33/00

(52) U.S. Cl. 435/6; 436/86

(57) ABSTRACT

The invention relates to a method for the early non-invasive diagnosis of fetal aneuploidy. In particular, the invention concerns the diagnosis of fetal aneuploidy by identifying protein expression patterns characteristics of fetal aneuploidy in a maternal biological fluid, such as maternal serum or amniotic fluid.

### BACKGROUND OF THE INVENTION

1. Field of the Invention

The present invention relates to a method for the early non-invasive diagnosis of fetal aneuploidy. In particular, the invention concerns the diagnosis of fetal aneuploidy by identifying protein expression patterns characteristics of aneuploidy in a maternal biological fluid, such as maternal serum or amniotic fluid.

2. Description of the Related Art

Proteomics

The large-scale analysis of protein expression patterns is emerging as an important and necessary complement to current DNA cloning and gene profiling approaches (Pandey and Mann, Nature 405:837-46 (2000)). DNA sequence information is helpful in deducing some structural and potential protein modifications based on homology methods, but it does not provide information on regulation of protein function through post-translational modifications, proteolysis or compartmentalization.

Traditional gel-based methods, such as one- and two-dimensional gel electrophoresis are useful for small-scale protein detection (<1,000 proteins), but these require large sample quantity (Lilley K S, Razzaq A, Dupree P: Two-dimensional gel electrophoresis: recent advances in sample preparation, detection and quantitation. Curr Opin Chem Biol. 6(1):46-50, 2002). Approaches to overcome this limitation include matrix-assisted or surface-enhanced laser desorption/ionization (MALDI or SELDI) time-of-flight mass spectrometers that accurately generate profiles showing the masses of proteins in a sample. These patterns or profiles can be used to identify and monitor various diseases. The second level of identification comes from coupling peptide mapping to tandem mass spectrometry to generate amino acid sequence information from peptide fragments. This can, for example, be achieved by coupling the MALDI/SELDI or ESI to quadrupole time-of-flight MS (Qq-TOF MS). The latter method can also be used for quantification of specific peptides (ICAT technology).

Fetal Aneuploidy

Fetal aneuploidies are aberrations in chromosome number and commonly arise as a result of a meiotic nondisjunction during oogenesis or spermatogenesis, however certain aneuploidies, such as trisomy 8, result more often from postzygotic mitotic disjunction (Nicolaidis & Petersen, Human Reproduction, 13(2):313-319, (1998)). Such abberations include both reductions and increases in the normal chromosome number and can involve autosomes as well as the sex chromosmes. An example of a reduction aneupolidy is Turner's syndrome, which is typified by the presence of a single X sex chromosome. Examples of increases in chromosome number include Down's syndrome (trisomy of chromosome 21), Patau syndrome (trisomy of chromosome 13), Edwards syndrome (trisomy of chromosome 18), and Kleinfelter's syndrom (an XXY trisomy of the sex chromosomes). Aneuploidies commonly lead to significant physical and neurological impairments which result in a large percentage of affected individuals failing to reach adulthood. In fact, fetuses having an autosomal aneuploidy involving a chromosome other than 13, 18, or 21 generally die in utero. However, certain aneuploidies, such as Kleinfelter's syndrome, present far less pronounced phenotypes and those affected with other trisomies, such as XXY & XXX, often will mature to be fertile adults.

Down's syndrome is the most common single pattern of malformation in man, and is one of the most common serious congenital abnormalities found at birth, with a prevalence of one in 660 live births (Jones, K., Down's Syndrome in Smith's recognizable patterns of human malformation, Jones, K., Editor, 1997, Philadelphia, Pa., pp. 8-13). Approximately a third of all fetuses with Down's syndrome who are alive in the second trimester will not survive to term; thus, the true prevalence of Down's syndrome in the second trimester is closer to 1 in 500 pregnancies (Cuckle, H., Epidemiology of Down Syndrome, in Screening for Down Syndrome in the First Trimester, J. Grudzinkas and R. Ward, Editors, 1997, RCOG Press, London, UK, pp. 3-13.). A majority of infants with Down's syndrome have serious cardiac, gastrointestinal, or other abnormalities that lead to significant morbidity and mortality. In addition, most have an IQ of less than 50, making this syndrome one of the leading causes of mental deficiency in the United States. Approximately 2.5 million pregnant women undergo serum screening for Down's syndrome each year in the United States, and, in the absence of screening, approximately 4,000 of these pregnancies may result in birth of a baby with Down's syndrome (Palomaki, G. E., et al. Am. J. Obstet. Gynecol. 176(5):1046-1051 (1997)).

While Down's syndrome is the most prevalent aneuploidy in live births, aneuploidies of chromosomes 13, 18, and the sex chromosomes affect a significant number of individuals. Trisomy 18, for example, has a prevelance of approximately 1 in 7000 births and Trisomy 13 has a prevalence of approximately 1 in 29,000 births (Nicolaidis & Petersen, supra). Other aneuploidies occur at significant rates during pregnancy, but result in spontaneous abortion before the fetus reaches term, usually within the first 15 weeks of pregnancy (Nicolaidies & Petersen, supra). For example, Trisomy 16 is single most prevelant human trisomy and is thought to affect 1.5% of all recognized pregnancies, however it is a lethal chromosomal abberation (Nicolaidies & Petersen, supra). Trisomies 15 and 8 occur at much lower rates (approximately 1.4% and 0.7% of all sponateous abortions, respectively) but are also lethal aberrations (Nicoladies & Petersen, supra).

Diagnosis of Fetal Aneuplody

Definitive prenatal diagnosis of fetal aneuploidies requires invasive testing by amniocentesis or Chorionic Villus Sampling (CVS), which are associated with a 0.5% to 1% procedure-related risk of pregnancy loss (D'Alton, M. E., Semin Perinatol 18(3):140-62 (1994)). Screening for fetal aneuploidies, such as Down's syndrome, is commonly performed during pregnancy to provide patients an assessment of their risk of carrying an affected fetus. Due to the risks associated with these invasive testing methods, much interest has developed in noninvasive methods of screening for aneuploidy.

While different approaches have been employed in connection with specific aneuploidies, in the case of Down's syndrome, screening was initially based entirely on maternal age, with an arbitrary cut-off of 35 years used to define a population of women at sufficiently high risk to warrant offering invasive fetal testing. This approach results in a detection rate of 20% to 30% of fetuses with Down's syndrome, with a 5% to 7% invasive fetal testing rate. Therefore, approximately 140 amniocenteses are required to detect each case of Down's syndrome, and one normal fetus is lost for every two affected fetuses detected (Vintzielos and Egan, Am J. Obstet Gynecol 172(3):837-44 (1995)).

Because of these limitations, second-trimester serum screening techniques were introduced in order to improve detection rate and to reduce the invasive testing rate. Current standard-of-care for screening for Down's syndrome requires offering all patients a triple-marker serum test between 15 and 18 weeks gestation, which, together with maternal age (MA), is used for risk calculation. This test assays (α-fetoprotein (AFP), human chorionic gonadotropin (βhCG), and unconjugated estriol (uE3). If the risk derived from this “triple screen” is greater than a predetermined cut-off, the patient is offered invasive testing for fetal karyotype analysis. The most commonly used risk cut-off is 1 in 380 (the term risk of a 35-year-old woman), which results in a 65% to 70% detection rate for Down's syndrome, with 5% to 7% of the pregnant population offered invasive fetal testing (Wald et al., J Med Screen 4(4):181-246 (1997)). It is estimated that 60 amniocenteses are performed to detect one case of Down's syndrome, using MA combined with this second trimester serum “triple screen” (Vintzielos and Egan, supra).

The current standard-of-care serum “triple screen” for Down's syndrome is now evolving into a “quad test”, in which the serum marker inhibin-A is added to the other three analytes. The quad test has been offered clinically since August 1996 at the Wolfson Institute of Preventive Medicine in London, under the direction of Professor Nicholas Wald. The performance of inhibin-A in everyday practice has been as predicted. Estimates of the performance of inhibin-A as a screening marker have been very consistent. In six published studies, maternal serum inhibin-A levels in cases of Down's syndrome pregnancy were, on average, 1.9-fold greater than those found in unaffected pregnancies (Wald et al., 1997, supra). It has been estimated that inhibin-A is almost as good as the most powerful single marker, βhCG, as a univariate predictor of a Down's syndrome pregnancy (at a fixed 5% screen-positive rate, inhibin-A has a 44% detection rate compared with a 49% detection rate for βhCG) (Wald et al., 1997, supra). The addition of inhibin-A to the triple test may improve the Down's syndrome detection rate of the “triple screen” to 77% to 80%, for a 5% to 7% invasive testing rate (Wald et al., 1997 supra; Wald et al., Prenat Diagrn 16(2):143-53 (1996)). Alternatively, the quad test may be used to maintain a 70% detection rate for Down's syndrome, while reducing the invasive testing rate to 5%, and significantly reducing the number of amniocenteses performed.

In an effort to reduce further the frequency of amniocenteses, second-trimester screening ultrasonography has been applied to Down's syndrome screening. The identification of certain major fetal structural abnormalities significantly increases the risk of Down's syndrome and other aneuploidies, and is then considered an indication for invasive fetal testing. However, this approach does not improve population screening for Down's syndrome, since 98% of fetuses in the general population do not have structural abnormalities.

Further work has been performed evaluating the role of sonographic markers of aneuploidy, which are not structural abnormalities per se, and, in the presence of a normal karyotype, may not confer any risks to the fetus. Such sonographic markers employed in Down's syndrome screening include choroid plexus cysts, echogenic bowel, short femur, short humerus, minimal hydronephrosis, and thickened nuchal fold. While some investigators have suggested that a sonographic approach may identify up to 73% of fetuses with Down's syndrome for a 5% screen-positive rate, these studies have all been derived from populations already at high risk for aneuploidy (Benacerraf et al., Radiology 193(l):135-40 (1994)). It is impossible to accurately extrapolate the performance of these tests from high-risk populations to general or unselected populations since the prevalence of the diseases in question will be significantly reduced. The value of this “genetic sonogram” is, therefore, severely limited when applied to screening of the general population. In addition, because of the subtlety of the findings, the performance of sonographic methods of screening are extremely dependent on the skill and experience of the operator, which may not be reproducible when sonographic screening is applied outside of tertiary centers (Ewigman, B. G., et al., N Engl J Med 329(12):821-7 (1993)). Although the “genetic sonogram” does not appear to be useful as a primary screening tool, it may have a role in reducing the risk of aneuploidy following an initial positive screening test (Vintzielos and Egan, supra).

A major problem with second-trimester screening for Down's syndrome is that it is performed at 15 to 18 weeks gestation, with diagnostic amniocentesis subsequently performed, if indicated, at 16 to 20 weeks gestation. This leads to significant time pressure on patients and providers if termination of pregnancy is desired before the commonly used upper gestational age limit of 24 weeks is reached. In addition, such later pregnancy terminations are associated with increased maternal morbidity (Lawson, H. W., et al., Am J. Obstet Gynecol 171(5):1365-72 (1994)). The value of a sonographic aneuploidy screening program based in the first trimester would include safe methods of pregnancy termination if an abnormality is confirmed, as well as improvement in patient privacy and confidentiality if abnormalities are detected at an early stage of pregnancy.

Investigators from the Fetal Medicine Foundation in London have suggested an 80% detection rate for Down's syndrome from screening using a combination of MA and first-trimester ultrasound evaluation of the fetus (Pandya, P. P. et al., Br J Obstet Gyneacol 102(12):957-62 (1995); Snijders, R. J., et al., Lancet 352(9125):343-6 (1998)). This relies on the measurement of the translucent space between the back of the fetal neck and overlying skin, which has been reported to be increased in fetuses with Down's syndrome and other aneuploidies. This nuchal translucency (NT) measurement is reportedly easy to obtain by transabdominal or transvaginal ultrasonography between 10 and 14 weeks gestation (Snijders, R. J., et al., Ultrasound Obstet Gynecol 7(3):216-26 (1996)). The vast majority of data supporting first-trimester screening for Down's syndrome is from the Fetal Medicine Foundation in London (Pandya et al., 1995, supra; Snijders et al., 1996, supra). However, the detection rates for Down's syndrome have not been consistent between different centers and, to date, no center outside of the Fetal Medicine Foundation network has been able to replicate their results.

There are also data suggesting that first-trimester concentrations of a variety of pregnancy-associated proteins and hormones differ in chromosomally normal and abnormal pregnancies. The two most promising first-trimester serum markers with regards to Down's syndrome and Edwards syndrome appear to be PAPP-A and free βhCG (Wapner, R., et al., N Engl J Med 349(15):1405-1413 (2003)). It has been reported that first-trimester serum levels of PAPP-A are significantly lower in Down's syndrome, and this decrease is independent of nuchal translucency (NT) thickness (Brizot, M. L., et al., Obstet Gynecol 84(6):918-22 (1994)). In addition, it has been shown that first-trimester serum levels of both total and free β-hCG are higher in fetal Down's syndrome, and this increase is also independent of NT thickness (Brizot, M. L., Br J Obstet Gynaecol 102(2):127-32 (1995)). PAPP-A and free βhCG are also independent of each other when applied to Down's syndrome screening (Wald and Hackshaw, Prenat Diagn 17(9):921-9 (1997)). In a multicenter prospective study, the combination of PAPP-A and free βhCG resulted in a 60% detection rate for Down's syndrome, for a 5% invasive testing rate (Haddow, J. E., et al., N Eng J Med 338(14):955-61 (1998)). Mathematical models have suggested that a combined first-trimester screening program utilizing MA, NT thickness, serum free βhCG, and serum PAPP-A will detect more than 80% of fetuses with Down's syndrome for a 5% invasive testing rate (Wald and Hackshaw, supra). These trials and models have recently been reviewed by Nicolaides (Ultrasound in Obstretics and Gynecology 21:313-21 (2003)).

While these data suggest that a combination first-trimester screening program or an integrated first and second-trimester screening program for fetal aneuploidies, such as Down's syndrome, would be superior to standard second-trimester screening, this hypothesis has not been validated in clinical practice.

To define the efficacy of first-trimester screening for Down's syndrome, and to compare the diagnostic performances of first and second-trimester screening, the NIH recently sponsored a multi-center First and Second Trimester Evaluation of Risk (FASTER) trial. In this prospective study, patients underwent an ultrasound for NT and had maternal serum obtained for PAPP-A and free βhCG at 10 3/7 -13 6/7 weeks of gestation, and results were blinded from patients until after a second risk screening at 15 - 18 6/7 weeks of gestation, which included a quad screen (AFP, βhCG, uE3, and inhibin-A). Over 38,000 patients completed the study, from which 117 cases of fetal trisomy-21 were identified, 87 of which had complete first and second-trimester data. The diagnostic performance of each test was analyzed by screening method, including: combined first-trimester screen (NT/PAPP-A/free βhCG/MA); second-trimester serum screen (maternal AFP/free βhCG/uE3/inhibin-A/MA); or integrated first and second-trimester screen.

While these data confirm the utility of first-trimester, or combined first and second-trimester integrated screening, there are important limitations. First, these tests are highly dependent upon gestational age, and become less discriminatory as gestation advances. Secondly, to optimize the detection of Down's syndrome, all of these tests have low screen-positive rates (5%) and extraordinarily high true false-positive rates (in excess of 90%), resulting in patient anxiety and unnecessary invasive amniocentesis for genetic testing. Thus, there is an urgent need for alternative tests that are reliable and robust across a wide range of gestational ages and that have a lower rate of false positives.

It is particularly desirable to develop new, efficient and reliable non-invasive methods for the diagnosis of Down's syndrome as well as other fetal aneuploidies.

### SUMMARY OF THE INVENTION

In one aspect the invention concerns a method for diagnosis of fetal aneuploidy, comprising comparing the proteomic profile of a test sample of a maternal biological fluid with a normal or a reference proteomic profile of the same type of biological fluid, and determining the presence of fetal aneuploidy if the proteomic profile of said test sample shows at least one unique expression signature representing at least one biomarker selected from the group consisting of the biomarkers listed in Tables 1-2 and 5-6, absent from said normal proteomic profile or present in said reference proteomic profile.

In an additional aspect, the invention concerns a method for diagnosis of fetal aneuploidy, comprising comparing the proteomic profile of a test sample of a maternal biological fluid with a normal or a reference proteomic profile of the same type of biological fluid, and determining the presence of fetal aneuploidy if the proteomic profile of said test sample shows at least one unique expression signature representing at least one biomarker selected from the group consisting of the biomarkers listed in Table 3, absent from said normal proteomic profile or present in said reference proteomic profile.

In one embodiment, the invention concerns the use of a test sample obtained from a pregnant female human.

In another embodiment of the invention, the proteomic profile is a mass spectrum.

In an additional embodiment of the invention, the test sample is maternal serum.

In another embodiment, the unique expression signature is in one or more of molecular weight regions 16 to 20 kDa, 35 to 38 kDa, 38 to 42 kDa, 40 to 45 kDa, 50 to 55 kDa, 60 to 68 kDa, and 125 to 150 kDa.

In another embodiment, the test sample is maternal amniotic fluid.

In another embodiment, the unique expression signature is in one or both of molecular weight regions of 6 to 7 kDa and 8 to 10 kDa.

In another embodiment, the method is performed in the first trimester of pregnancy.

In another embodiment, the method is performed in the second trimester of pregnancy.

In an additional embodiment, the method further comprises determining the level of transcribed mRNA or the level of translated protein of at least one biomarker of fetal aneuploidy in the test sample, and confirming the presence of fetal aneuploidy if said level of transcribed mRNA or level of translated protein is different relative to its level in a normal biological sample.

In another embodiment, The fetal aneuploidy being diagnosed is Down's syndrome, trisomy 13, trisomy 18, X chromosome trisomy, X chromosome monosomy, Kleinfelter's syndrome (XXY genotype), or XYY syndrome (XYY genotype).

In another embodiment, the biomarker whose level of transcribed mRNA or level of translated protein is being detected is selected from the group consisting of PAPP-A, a-fetoprotein (AFP), human chorionic gonadotropin (bhCG), unconjugated estriol (uE3), and inhibin A.

In an additional embodiment, The method further comprising subjecting the pregnant female human to one or more of additional diagnostic techniques.

In another embodiment, the additional diagnostic techniques are selected from the group consisting of ultrasonography, techniques to test chromosomal abnormalities, and nuchal translucency (NT) measurement.

In an additional embodiment, the invention involves that comparison of the unique expression signature of more than one biomarker. In additon, the number of expression signatures can be of 2, 3, 4, 5, 6, 7, 8, or more biomarkers.

In an additional embodiment the biomarker or biomarkers are selected from the group consisting of complement factor H (CFAH_HUMAN, SwissProt Accession No. P08603); pregnancy zone protein (PZP_HUMAN; SwissProt Accession No. P20741); afamin (AFAM_HUMAN; SwissProt Accession No. P43652); angiotensinogen (ANGT_HUMAN; SwissProt Accession No. P01019); alpha-2-hs-glycoprotein (A2HS_HUMAN; SwissProt Accession No. P02765); clusterin (CLUS_HUMAN; SwissProt Accession No. P10909); apolipoprotein AI (APA1_HUMAN; SwissProt Accession No. P02647); apolipoprotein AIV (APA4_HUMAN; SwissProt Accession No. P06727); apolipoprotein E (APE_HUMAN; SwissProt Accession No. P02649); pigment epithelium-derived factor (PEDF_HUMAN; SwissProt Accession No. P36955); serum amyloid A protein (SAA_HUMAN; SwissProt Accession No. P02735); AMBP protein (AMBP_HUMAN; SwissProt Accession No. P02760); plasma retinol binding protein (RETB_HUMAN; SwissProt Accession No. P02753); serotransferrin precursor (TRFE_HUMAN; SwissProt Accession No. P02787); alpha-1-antitrypsin precursor (A1AT_HUMAN; SwissProt Accession No. P01009); alpha-2-macroglobulin precursor (A2MG_HUMAN; SwissProt Accession No. P01023); complement C3 precursor (CO3_HUMAN; SwissProt Accession No. P01024); angiotensinogen precursor (ANGT_HUMAN; SwissProt Accession No. P01019); ceruloplasmin precursor (CERU_HUMAN; SwissProt Accession No. P00450); haptoglobin precursor (HPT_HUMAN; SwissProt Accession No. P00738); antithrombin-III precursor (ANT3_HUMAN; SwissProt Accession No. P01008); hemopexin precursor (HEMO_HUMAN; SwissProt Accession No. P02790); alpha-1-acid glycoprotein 1 precursor (A1AG_HUMAN; SwissProt Accession No. P02763); apolipoprotein A-I precursor (APA1_HUMAN; SwissProt Accession No. P02647); alpha 1b-glycoprotein (SwissProt Accession No. P04217); kininogen precursor (KNG_HUMAN; SwissProt Accession No. P01042-2); inter-alpha-trypsin inhibitor heavy chain H2 precursor (ITH2_HUMAN; SwissProt Accession No. P19823); alpha-2-hs-glycoprotein precursor (A2HS_HUMAN; SwissProt Accession No. P02765); alpha-1-antichymotrypsin precursor (AACT_HUMAN; SwissProt Accession No. P01011); inter-alpha-trypsin inhibitor heavy chain H4 precursor (ITH4_HUMAN; SwissProt Accession No. Q14624-2); complement factor H precursor (CFAH_HUMAN; SwissProt Accession No. P08603-1); plasma protease C1 inhibitor precursor (IC1_HUMAN; SwissProt Accession No. P05155); heparin cofactor II precursor (HEP2_HUMAN SwissProt Accession No. P05546); complement factor B precursor (CFAB_HUMAN; SwissProt Accession No. P00751-1); alpha-2-glycoprotein 1, zinc (ZA2G_HUMAN; SwissProt Accession No. P25311); vitronectin precursor (VTNC_HUMAN SwissProt Accession No. P04004); inter-alpha-trypsin inhibitor heavy chain H1 precursor (ITH1_HUMAN; SwissProt Accession No. P19827); complement component C9 precursor (CO9_HUMAN; SwissProt Accession No. P02748); fibrinogen alpha/alpha-E chain precursor (FIBA_HUMAN; SwissProt Accession No. P02671-1); fibrinogen beta chain precursor (FIBB_HUMAN; SwissProt Accession No. P02675); fibrinogen gamma chain precursor (FIBG_HUMAN; SwissProt Accession No. P02679-1); prothrombin precursor (THRB_HUMAN; SwissProt Accession No. P00734); clusterin precursor (CLUS_HUMAN; SwissProt Accession No. P10909); alpha-1B-glycoprotein precursor (A1BG_HUMAN; SwissProt Accession No. P04217); alpha-1-acid glycoprotein 2 precursor (A1AH_HUMAN; SwissProt Accession No. P19652); apolipoprotein D precursor (APOD_HUMAN; SwissProt Accession No. P05090); pregnancy zone protein precursor (PZP_HUMAN; SwissProt Accession No. P20742); histidine-rich glycoprotein precursor (HRG_HUMAN; SwissProt Accession No. P04196); sex hormone-binding globulin precursor (SHBG_HUMAN; SwissProt Accession No. P04278-1); plasminogen precursor (PLMN_HUMAN; SwissProt Accession No. P00747); apolipoprotein C-III precursor (APC3_HUMAN; SwissProt Accession No. P02656); leucine-rich alpha-2-glycoprotein precursor (A2GL_HUMAN; SwissProt Accession No. P02750); apolipoprotein E precursor (APE_HUMAN; SwissProt Accession No. P02649); fetuin-B precursor (FETB_HUMAN; SwissProt Accession No. Q9UGM5); myosin-reactive immunoglobulin light chain variable region (SwissProt Accession No. Q9UL83); complement C1S component precursor (C1S_HUMAN; SwissProt Accession No. P09871); ambp protein precursor (AMBP_HUMAN; SwissProt Accession No. P02760); and complement C4 precursor (CO4_HUMAN; SwissProt Accession No. P01028).

In a particular embodiment, the biomarkers employed in the invention are complement factor H (CFAH_HUMAN, SwissProt Accession No. P08603); and pregnancy zone protein (PZP_HUMAN; SwissProt Accession No. P20741).

In a particular embodiment, the biomarkers employed in the invention are complement factor H (CFAH_HUMAN, SwissProt Accession No. P08603); and afamin (AFAM_HUMAN; SwissProt Accession No. P43652).

In a particular embodiment, the biomarkers employed in the invention are pregnancy zone protein (PZP_HUMAN; SwissProt Accession No. P20741); and alpha-2-hs-glycoprotein (A2HS_HUMAN; SwissProt Accession No. P02765).

In a particular embodiment, the biomarkers employed in the invention are complement factor H (CFAH_HUMAN, SwissProt Accession No. P08603); angiotensinogen (ANGT_HUMAN; SwissProt Accession No. P01019); and clusterin (CLUS_HUMAN; SwissProt Accession No. P10909).

In a particular embodiment, the biomarkers employed in the invention are apolipoprotein E (APE_HUMAN; SwissProt Accession No. P02649); AMBP protein (AMBP_HUMAN; SwissProt Accession No. P02760); and plasma retinol binding protein (RETB_HUMAN; SwissProt Accession No. P02753).

In a particular embodiment, the biomarkers employed in the invention are complement factor H (CFAH_HUMAN, SwissProt Accession No. P08603); afamin (AFAM_HUMAN; SwissProt Accession No. P43652); angiotensinogen (ANGT_HUMAN; SwissProt Accession No. P01019); and clusterin (CLUS_HUMAN; SwissProt Accession No. P10909).

In a particular embodiment, the biomarkers employed in the invention are complement factor H (CFAH_HUMAN, SwissProt Accession No. P08603); afamin (AFAM_HUMAN; SwissProt Accession No. P43652); pigment epithelium-derived factor (PEDF_HUMAN; SwissProt Accession No. P36955); serum amyloid A protein (SAA_HUMAN; SwissProt Accession No. P02735); angiotensinogen (ANGT_HUMAN; SwissProt Accession No. P01019); and clusterin (CLUS_HUMAN; SwissProt Accession No. P10909).

In a particular embodiment, the biomarkers employed in the invention are apolipoprotein E (APE_HUMAN; SwissProt Accession No. P02649); AMBP protein (AMBP_HUMAN; SwissProt Accession No. P02760); plasma retinol binding protein (RETB_HUMAN; SwissProt Accession No. P02753); serotransferrin precursor (TRFE_HUMAN; SwissProt Accession No. P02787); alpha-2-macroglobulin precursor (A2MG_HUMAN; SwissProt Accession No. P01023); and histidine-rich glycoprotein precursor (HRG_HUMAN; SwissProt Accession No. P04196).

In a particular embodiment, the biomarkers employed in the invention are inter-alpha-trypsin inhibitor heavy chain H1 precursor (ITH1_HUMAN; SwissProt Accession No. P19827); complement component C9 precursor (CO9_HUMAN; SwissProt Accession No. P02748); fibrinogen alpha/alpha-E chain precursor (FIBA_HUMAN; SwissProt Accession No. P02671-1); apolipoprotein C-III precursor (APC3_HUMAN; SwissProt Accession No. P02656); leucine-rich alpha-2-glycoprotein precursor (A2GL_HUMAN; SwissProt Accession No. P02750); apolipoprotein E precursor (APE_HUMAN; SwissProt Accession No. P02649); fetuin-B precursor (FETB_HUMAN; SwissProt Accession No. Q9UGM5); and complement C4 precursor (CO4_HUMAN; SwissProt Accession No. P01028).

In a particular embodiment, the inventions involves the use of proteomic profiles that include at least one glycoprotein.

In a particular embodiment, the invention involves the glycoprotein or glycoproteins employed in the proteomic profile are selected from the group consisting of sialic acid glycoproteins, mannose binding glycoproteins, and O-linked glycoproteins.

In a particular embodiment, the invention involves the detection of a fetal aneuploidy that is an autosomal aneuploidy.

In an additional embodiment, the invention involes the detection of a trisomy of chromosomes 13, 18, or 21.

In a particular embodiment, the invention involves the detection of a fetal aneuploidy that is a sex chromosome aneuploidy.

In an additional embodiment, the invention involes the detection of an aneuploidy selected from the group consisting of: X chromosome trisomy, X chromosome monosomy, Keinfelter's syndrome (XXY genotype), and XYY syndrome (XYY genotype).

### BRIEF DESCRIPTION OF THE DRAWINGS

Table 1. Candidate maternal serum biomarkers in Down's syndrome, identified from the initial 7 areas of interest (FIG. 2). Tandem MS/MS analysis of the ingel digests of 2D spots followed by de novo sequencing and database search using OpenSea revealed the relative abundance of each protein in these areas.

Table 2. Candidate maternal serum biomarkers in Down's syndrome identified. Tandem MS/MS analysis of the ingel digests of 2D spots followed by de novo sequencing and database search using OpenSea revealed the relative abundance of each protein in these areas.

Table 3. Candidate amniotic fluid biomarkers in Down's syndrome identified. Tandem MS/MS analysis of the ingel digests of 2D spots followed by de novo sequencing and database search using OpenSea revealed the relative abundance of each protein in these areas.

Table 4. Preferred maternal serum and amniotic fluid biomarkers for diagnosis of fetal Down's syndrome.

Table 5. Candidate maternal serum biomarkers in Down's syndrome, identified from the initial areas of interest (FIG. 7). Tandem MS/MS was employed to identify the specific candidate biomarkers.

Table 6. Candidate maternal serum biomarkers in Down's syndrome, identified from the initial areas of interest (FIGS. 8-11). Tandem MS/MS was employed to identify the specific candidate biomarkers.

FIG. 1. SELDI-TOF-MS analysis of maternal serum from 2^{nd} trimester Control and Down's samples. Top panel represents pooled control from all 4 matched cases. Area of interest was boxed showing a potential peak that is differentially expressed between the two groups.

FIG. 2. 2-D gels of maternal serum samples (20 μg of protein) purified using Agilent immunoaffinity columns labeled with 100 pm of Cus5 (Down's syndrome) or Cy3 (Control). Gels were scanned at 600 PMT voltage in a Typhoon 94100 Scanner (Amersham Biosciences). Images overlaid using Phoretic 2D Evolution (nonlinear Dynamics).

FIG. 3. Immuno-MALDI-TOF-MS assay. Spectra of immunoprecipitated apolipoproteins A). apolipoprotein A1. B). apolipoprotein A2. C). apolipoprotein E from maternal control (blue trace) and Down's (red trace) serum. Panel D is an inset taken from the 2D DIGE gel in FIG. 2 from which several apolipoprotein species were identified by tandem mass spectrometry.

FIG. 4. Detection of differential protein expression in maternal serum. 2-D western immunolbots probed with human complement factor H antibodies. A) control serum 2nd trimester; B) Down's syndrome maternal serum 2nd trimester.

FIG. 5. Schematic representation of de novo protein sequence identification of candidate biomarkers in Down's syndrome. Spectra representing peptide sequences that belong to Complement factor H.

FIG. 6. Schematic representation of de novo protein sequence identification of candidate biomarkers in Down's syndrome. Sequence coverage map of peptide sequences identified that belong to Complement factor H. Lighter shading peptides identified, darker shading represent potential protein modifications of these amino acids.

FIG. 7. MS analysis of collected differential 2-D liquid chromatography fractions. A) The 2D-LC maps generated using ProteoVue software display the p1 of the eluted protein from CF on the x-axis and the retention time, or hydrophobicity, of the eluted protein from RP-HPLC on the y-axis. B) the 2D map of the control sample is depicted in red on the left and the 2D map of the DS sample is depicted in green on the right. The center of the figure displays the difference map (displayed separately in B) of the two samples, where bands seen in green are proteins up-regulated in the DS sample and bands seen in red are proteins up-regulated in the control sample.

FIG. 8. Fluorescent 2-dimensional gel image representing differential expression of total glycoproteins in second trimester Control (Red) and DS (Green) maternal serum.

FIG. 9. Fluorescent 2-dimensional gel image representing differential expression of Sialic-glycoproteins in second trimester Control (Red) and DS (Green) maternal serum.

FIG. 10. Fluorescent 2-dimensional gel image representing differential expression of Mannose binding glycoproteins in second trimester Control (Red) and DS (Green) maternal serum.

FIG. 11. Fluorescent 2-dimensional gel image representing differential expression of O-linked glycoproteins in second trimester Control (Red) and DS (Green) maternal serum.

FIG. 12. MALDI-TOF of total glycoproteins trypsin digest. Maternal serum of control (top) and Down's syndrome (bottom). Significant differences in peptides expressed in Down's syndrome are boxed.

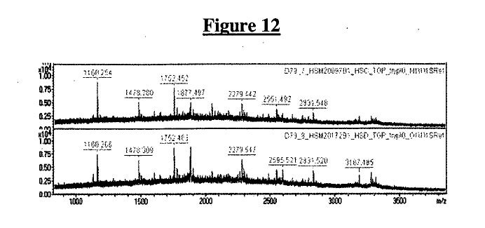

FIG. 13. MALDI-TOF of Sialic acid glycoproteins trypsin digest. Maternal serum of control (top) and Down's syndrome (bottom). Significant differences in peptides expressed in Down's syndrome are boxed.

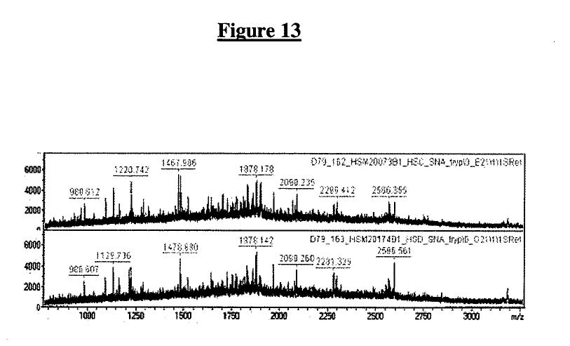

FIG. 14. MALDI-TOF of Mannose binding glycoproteins trypsin digest. Maternal serum of control (top) and Down's syndrome (bottom). Significant differences in peptides expressed in Down's syndrome are boxed.

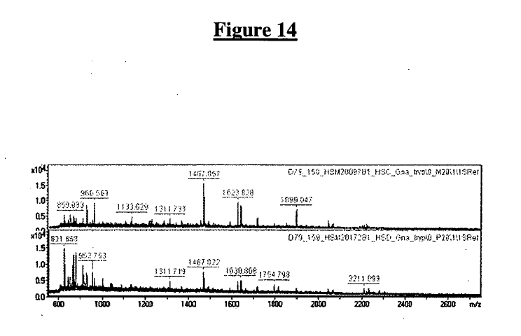

FIG. 15. MALDI-TOF of O-linked glycoproteins trypsin digest. Maternal serum of control (top) and Down's syndrome (bottom). Significant differences in peptides expressed in Down's syndrome are boxed.

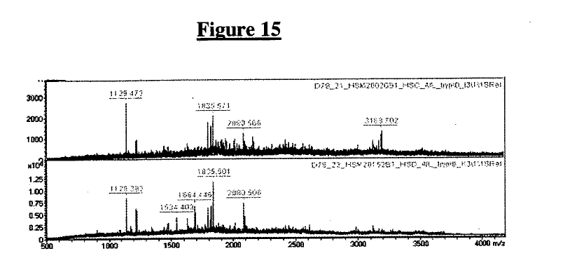

FIG. 16. 2-D gels of maternal serum samples (20 μg of protein) purified using Agilent immunoaffinity columns labeled with 100 pm of Cus5 (Trisomy 18) or Cy3 (Control). Gels were scanned at 600 PMT voltage in a Typhoon 94100 Scanner (Amersham Biosciences). Images overlaid using Phoretic 2D Evolution (nonlinear Dynamics).

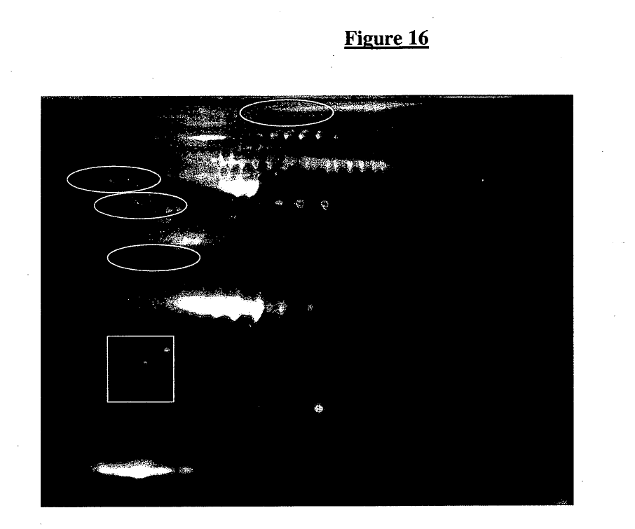

FIG. 17. 2-D gels of maternal serum samples (20 μg of protein) purified using Agilent immunoaffinity columns labeled with 100 pm of Cus5 (Trisomy 13) or Cy3 (Control). Gels were scanned at 600 PMT voltage in a Typhoon 94100 Scanner (Amersham Biosciences). Images overlaid using Phoretic 2D Evolution (nonlinear Dynamics).

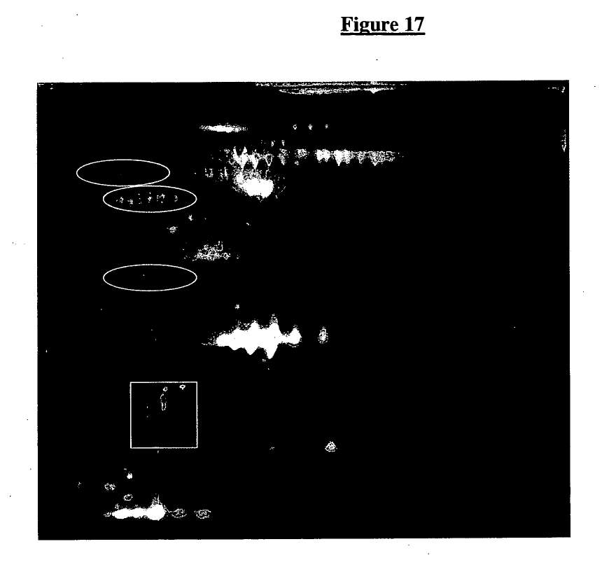

FIG. 18. 2-D gels of maternal serum samples (20 μg of protein) purified using Agilent immunoaffinity columns labeled with 100 pm of Cus5 (Neural Tube Defects) or Cy3 (Control). Gels were scanned at 600 PMT voltage in a Typhoon 94100 Scanner (Amersham Biosciences). Images overlaid using Phoretic 2D Evolution (nonlinear Dynamics).

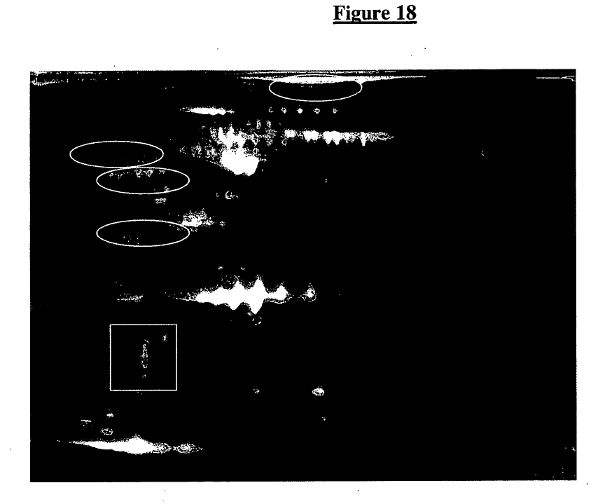

### DETAILED DESCRIPTION OF THE PREFERRED EMBODIMENT

A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994) provides one skilled in the art with a general guide to many of the terms used in the present application.

The term “proteome” is used herein to describe a significant portion of proteins in a biological sample at a given time. The concept of proteome is fundamentally different from the genome. While the genome is virtually static, the proteome continually changes in response to internal and external events.

The term “proteomic profile” is used to refer to a representation of the expression pattern of a plurality of proteins in a biological sample, e.g. a biological fluid at a given time. The proteomic profile can, for example, be represented as a mass spectrum, but other representations based on any physicochemical or biochemical properties of the proteins, or fragments thereof, are also included. Thus the proteomic profile may, for example, be based on differences in the electrophoretic properties of proteins, as determined by two-dimensional gel electrophoresis, e.g. by 2-D PAGE, and can be represented, e.g. as a plurality of spots in a two-dimensional electrophoresis gel. Alternatively, the proteomic profile may be based on differences in protein isolectric point and hydrophobicity, as determined by two-dimensional liquid chromatography, and can be represented, e.g. as a computer generated virtual two-dimensional map. Furthermore, lectin-based affinity purification can be combined with the techniques described herein to generate proteomic profiles that highlight the specific glycosylation properties of various proteins found in a biological sample.

Differential expression profiles may have important diagnostic value, even in the absence of specifically identified proteins. Single protein spots or chromatographic eluents can then be detected, for example, by immunoblotting, and multiple spots, eluents, or proteins can be identified using protein microarrays. The proteomic profile typically represents or contains information that could range from a few peaks to a complex profile representing 50 or more peaks. Thus, for example, the proteomic profile may contain or represent at least 2, or at least 3, or a least 4, or a least 5, or at least 6, or at least 7, or at least 8, or at least 9, or at least 10, or at least 15, or at least 20, or at least 25, or at least 30, or at least 35, or at least 40, or at least 45, or at least 50 proteins, and the like.

The term “unique expression signature” is used to describe a unique feature or motif within the proteomic profile of a biological sample (e.g. a reference sample or a test sample) that differs from the proteomic profile of a corresponding normal biological sample (obtained from the same type of source, e.g. biological fluid) in a statistically significant manner.

The term “normal proteomic profile” is used to refer to the proteomic profile of a biological sample of a maternal biological fluid of the same type as a test sample, that has been obtained from a pregnant female carrying a fetus not having an aneuploidy, or other chromosomal abnormality.

The term “reference proteomic profile” is used to refer to the proteomic profile of a biological sample of a maternal biological fluid of the same type as a test sample, that has been obtained from a pregnant female carrying a fetus having an aneuploidy.

“Patient response” can be assessed using any endpoint indicating a benefit to the patient, including, without limitation, (1) inhibition, at least to some extent, of the progression of a pathologic condition, (2) prevention of the pathologic condition, (3) relief, at least to some extent, of one or more symptoms associated with the pathologic condition; (4) increase in the length of survival following treatment; and/or (5) decreased mortality at a given point of time following treatment.

The term “treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

“Congenital malformation” is an abnormality which is non-hereditary but which exists at birth.

“Sensitivity” of a diagnostic assay or “diagnostic sensitivity” is defined as the probability of the test finding disease among those who have the disease, or proportion of people with disease who have a positive test result. In statistical terms: sensitivity=true positives/(true positives+false negatives).

The term “one or more” in the context of the proteomics profiles, protein markers, and unique expression signatures herein is used used mean any one, two, three, four, etc. of the listed members within a group, in any permutation. Accordingly, the term “one or more” includes any two, any three, any four, etc. of the members spepcifically listed within a group. While specific subgroups are listed throughout the specification and the claims, these are no limiting. It is emphasized that the term “one or more” is used in the broadest sense, and is used to designate any subgroup within a group with multiple members. Similarly, the terms “at least 2,” “at least 3,” “at least 4,” etc., cover any combinations of the members within a particular group, provided that the total number of members within the combination is at least 3, at least 3, at least, 4, etc.

B. Detailed Description

The present invention concerns methods and means for an early, reliable and non-invasive testing of fetal Down's syndrome and other chromosomal aneuploidies, based upon the proteomic profile of a maternal biological fluid. The invention utilizes proteomics techniques well known in the art, as described, for example, in the following textbooks, the contents of which are hereby expressly incorporated by reference: Proteome Research: New Frontiers in Functional Genomics (Principles and Practice), M. R. Wilkins et al., eds., Springer Verlag, 1007; 2-D Proteome Analysis Protocols, Andrew L Link, editor, Humana Press, 1999; Proteome Research: Two-Dimensional Gel Electrophoresis and Identification Methods (Principles and Practice), T. Rabilloud editor, Springer Verlag, 2000; Proteome Research: Mass Spectrometry (Principles and Practice), P. James editor, Springer Verlag, 2001; Introduction to Proteomics, D. C. Liebler editor, Humana Press, 2002; Proteomics in Practice: A Laboratory Manual of Proteome Analysis, R. Westermeier et al., eds., John Wiley & Sons, 2002.

One skilled in the art will recognize many methods and materials similar or equivalent to those described herein, which could be used in the practice of the present invention. Indeed, the present invention is in no way limited to the methods and materials described.

1. Identification of Proteins and Polypeptides Expressed in Biological Fluids

According to the present invention, proteomics analysis of biological fluids can be performed using a variety of methods known in the art.

Typically, protein patterns (proteome maps) of samples from different sources, such as normal biological fluid (normal sample) and a test biological fluid (test sample), are compared to detect proteins that are up- or down-regulated in a disease. These proteins can then be excised for identification and full characterization, e.g. using peptide-mass fingerprinting and/or mass spectrometry and sequencing methods, or the normal and/or disease-specific proteome map can be used directly for the diagnosis of the disease of interest, or to confirm the presence or absence of the disease.

In comparative analysis, it is important to treat the normal and test samples exactly the same way, in order to correctly represent the relative abundance of proteins, and obtain accurate results. The required amount of total proteins will depend on the analytical technique used, and can be readily determined by one skilled in the art. The proteins present in the biological samples are typically separated by two-dimensional gel electrophoresis (2-DE) according to their pI and molecular weight. The proteins are first separated by their charge using isoelectric focusing (one-dimensional gel electrophoresis). This step can, for example, be carried out using immobilized pH-gradient (IPG) strips, which are commercially available. The second dimension is a normal SDS-PAGE analysis, where the focused IPG strip is used as the sample. After 2-DE separation, proteins can be visualized with conventional dyes, like Coomassie Blue or silver staining, and imaged using known techniques and equipment, such as, e.g. Bio-Rad GS800 densitometer and PDQUEST software, both of which are commercially available. Individual spots are then cut from the gel, destained, and subjected to tryptic digestion. The peptide mixtures can be analyzed by mass spectrometry (MS).

Alternative methods of comparative analysis, and combinations of these various methods, may also be used within the scope of the instant invention. For example, proteins present in the biological samples may be separated by two-dimensional liquid chromatography according to their isoelectric point and hydrophobicity as described in Example II below. Of course, the chromatographic separation need not be based on hydrophobicity, as a wide range of separation materials are well known in the art including, but not limited to, materials capable of separation based on molecular weight, pH, or specific binding affinities such as antibody-antigen interactions. Furhthermore, once an initial separation step is complete, the peptides present in an individual spot or eluant sample can be separated by capillary high pressure liquid chromatography (HPLC) and can be analyzed by MS either individually, or in pools.

As detailed in Example III, glycosylation is an important posttranslational protein modifications in eukaryotes, and thus a system for separation and identification of the glycosylation state of a biological sample can be a valuable tool in mining protein biomarkers. Lectin based affinity purification is the method of choice for isolating different classes of glycosylated proteins due to their ability to specifically and reversibly bind to glycan moieties in glycoproteins. The major classes and types of glycoproteins can be individually isolated from the test samples and once separated, mass spectrometry can be employed to generate a differential glycosylation profile to compare control versus disease.

A discussed in detail below, a wide variety of lectins and their specificities are known in the art. One or more of these lectins, as well as any permutation of the possible combination of these and other lectins, can be used in practicing the instant invention. Mannose binding lectins are known to include, but are not limited to, the following: Concanavalin A from Canavalia ensiformis which binds branched α-mannosidic structures, high-mannose type, and hybrid type and biantennary complex type N-Glycans; Lentil lectin from Lens culinaris which binds the fucosylated core region of bi- and triantennary complex type N-Glycans; and Snowdrop lectin from Galanthus nivalis which binds a 1-3 and α 1-6 linked high mannose structures. Galactose/N-acetylgalactosamine binding lectins include, but are not limited to, the following: Ricinus communis Agglutinin (RCA₁₂₀) from Ricinus communis which binds Galβ1-4GlcNAcβ1-R; Peanut Agglutinin from Arachis hypogaea Galβ1-3GalNAcα1-Ser/Thr (T-Antigen); Jacalin from Artocarpus integrifolia which binds (Sia)Galβ1-3GalNAcα1-Ser/Thr (T-Antigen); and Hairy vetch lectin from Vicia villosa which binds GalNAcα-Ser/Thr (Tn-Antigen). Sialic acid/N-acetylglucosamine binding lectins include, but are not limited to, the following: Wheat Germ agglutinin from Triticum vulgaris which binds GlcNAcβ1-4GlcNAcβ1-4GlcNAc, and Neu5Ac (sialic acid); Elderberry lectin from Sambucus nigra which binds Neu5Acα2-6Gal(NAc)-R; Maackia amurensis lectin from Maackia amurensis which binds Neu5Ac/Gcα2-3Galβ1-4GlcNAcβ1-R. Fucose binding lectins include, but are not limited to, the following: Ulex europaeus agglutinin from Ulex europaeus which binds Fucα1-2Gal-R; Aleuria aurantia lectin from Aleuria aurantia which binds Fucα1-2Galβ1-4(Fucα1-3/4)Galβ1-4GlcNAc, and R2-GlcNAcβ1-4(Fucα1-6)GlcNAc-R1

Mass spectrometers consist of an ion source, mass analyzer, ion detector, and data acquisition unit. First, the peptides are ionized in the ion source. Then the ionized peptides are separated according to their mass-to-charge ratio in the mass analyzer and the separate ions are detected. Mass spectrometry has been widely used in protein analysis, especially since the invention of matrix-assisted laser-desorption ionisation/time-of-flight (MALDI-TOF) and electrospray ionisation (ESI) methods. There are several versions of mass analyzer, including, for example, MALDI-TOF and triple or quadrupole-TOF, or ion trap mass analyzer coupled to ESI. Thus, for example, a Q-Tof-2 mass spectrometer utilizes an orthogonal time-of-flight analyzer that allows the simultaneous detection of ions across the full mass spectrum range. For further details see, e.g. Chemusevich et al., J. Mass Spectrom. 36:849-865 (2001).

If desired, the amino acid sequences of the peptide fragments and eventually the proteins from which they derived can be determined by techniques known in the art, such as certain variations of mass spectrometry, or Edman degradation.

A method for determining sequences of molecules from mass spectrometry data is disclosed in co-pending application Ser. No. 10/789,424 filed on Feb. 27, 2004, the entire disclosure of which is hereby expressly incorporated by reference. The method involves de novo sequencing and database searching, and can also be used to identify sequence variations and unknown proteins, which have not been completely sequecnes but have close sequence homology to sequences present in sequence databases.

2. Chromosomal Aneuploidies

Chromosomal abnormalities are a frequent cause of perinatal morbidity and mortality. Chromosomal abnormalities occur with an incidence of 1 in 200 live births. The major cause of these abnormalities is chromosomal aneuploidy, an abnormal number of chromosomes inherited from the parents. One of the most frequent chromosomal aneuploidies is trisomy-21 (Down's syndrome), which has an occurrence of 1 in 800 livebirths (Hook E B, Hamerton J L: The frequency of chromosome abnormalities detected in consecutive newborn studies: Differences between studies: Results by sex and by severity of phenotypic involvement. In Hook E B, Porter I H (eds): Population Cytogenetics, pp 63-79. New York, Academic Press, 1978). The primary risk factor for trisomy-21 is maternal age greater than 35, but 80% of children with trisomy-21 are born to women younger than 35 years of age. Other common aneuploidic conditions include trisomies 13 and 18, Turner Syndrome and Klinefelter syndrome.

3. Diagnosis of Fetal Chromosomal Aneuploidies Using the Proteomic Profile of Biological Fluids or Biomarkers Identified in Biological Fluids

The present invention provides an early and reliable, non-invasive method for the diagnosis of fetal chromosomal aneuploidies base upon proteomic analysis of biological fluids, such as, for example, amniotic fluid, serum, plasma, urine, cerebrospinal fluid, breast milk, mucus, or saliva of a pregnant female.

As noted before, in the context of the present invention the term “proteomic profile” is used to refer to a representation of the expression pattern of a plurality of proteins in a biological sample, e.g. a biological fluid at a given time. The proteomic profile can, for example, be represented as a mass spectrum, but other representations based on any physicochemical or biochemical properties of the proteins are also included. Although it is possible to identify and sequence all or some of the proteins present in the proteome of a biological fluid, this is not necessary for the diagnostic use of the proteomic profiles generated in accordance with the present invention. Diagnosis can be based on characteristic differences (unique expression signatures) between a normal proteomic profile, and proteomic profile of the same biological fluid obtained under the same circumstances, when the chromosomal aneupliody to be diagnosed, such as Down's syndrome of the fetus, is present. The unique expression signature can be any unique feature or motif within the proteomic profile of a test or reference biological sample that differs from the proteomic profile of a corresponding normal biological sample obtained from the same type of source, in a statistically significant manner. For example, if the proteomic profile is presented in the form of a mass spectrum, the unique expression signature is typically a peak or a combination of peaks that differ, qualitatively or quantitatively, from the mass spectrum of a corresponding normal sample. Thus, the appearance of a new peak or a combination of new peaks in the mass spectrum, or any statistically significant change in the amplitude or shape of an existing peak or combination of existing peaks in the mass spectrum can be considered a unique expression signature. When the proteomic profile of the test sample obtained from a pregnant female subject is compared with the proteomic profile of a reference sample comprising a unique expression signature characteristic of a chromoromal aneuploidy the fetus is diagnosed with such chromosomal aneuploidy if the test sample shares the unique expression signature with the reference sample.

A particular chromosomal aneuploidy, such as fetal Down's syndrome, can be diagnosed by comparing the proteomic profile of a biological fluid obtained from the maternal subject tested, with the proteomic profile of a normal biological fluid of the same kind, obtained and treated the same manner. If the proteomic profile of the test sample is essentially the same as the proteomic profile of the normal sample, the fetus is considered to be free of the tested chromosomal aneuploidy. If the proteomic profile of the test sample shows a unique expression signature relative to the proteomic profile of the normal sample, the fetus is diagnosed with the chromosomal aneuploidy.

Alternatively or in addition, the proteomic profile of the test sample may be compared with the proteomic profile of a reference sample, obtained from a biological fluid of a pregnant female independently diagnosed with the condition in question. In this case, the fetus is diagnosed with the pathologic condition if the proteomic profile of the test sample shares at least one feature, or a combination of features representing a unique expression signature, with the proteomic profile of the reference sample.

In the methods of the present invention the proteomic profile of a normal biological sample plays an important diagnostic role. As discussed above, if the proteomic profile of the test sample is essentially the same as the proteomic profile of the normal biological sample, the fetus is diagnosed as being free of the chromosomal aneuploidy to be identified. The data are analyzed to determine if the differences are statistically significant.

The sensitivity of the diagnostic methods of the present invention can be enhanced by removing the proteins found both in normal and diseased proteome at essentially the same expression levels (common proteins, such as albumin and immunoglobulins) prior to analysis using conventional protein separation methods. The removal of such common proteins, which are not part of the unique expression signature, results in improved sensitivity and diagnostic accuracy. Alternatively or in addition, the expression signatures of the common proteins can be eliminated (or signals can be removed) during computerized analysis of the results, typically using spectral select algorithms, that are machine oriented, to make diagnostic calls. The results detailed in the Examples below present proteomic profiles characteristics of aneuploidies that differ from the normal proteomic profile of the maternal serum or amniotic fluid in a statistically significant manner. In addition, the Example and the enclosed Figures identify individual biomarkers, groups of biomarkers, and unique expression signatures characteristic of aneuploidies.

Statistical methods for comparing proteomic profiles are well known in the art. For example, in the case of a mass spectrum, the proteomic profile is defined by the peak amplitude values at key mass/charge (M/Z) positions along the horizontal axis of the spectrum. Accordingly, a characteristic proteomic profile can, for example, be characterized by the pattern formed by the combination of spectral amplitudes at given M/Z vales. The presence or absence of a characteristic expression signature, or the substantial identity of two profiles can be determined by matching the proteomic profile (pattern) of a test sample with the proteomic profile (pattern) of a reference or normal sample, with an appropriate algorithm. A statistical method for analyzing proteomic patterns is disclosed, for example, in Petricoin III, et al., The Lancet 359:572-77 (2002).; Issaq et al., Biochem Biophys Commun 292:587-92 (2002); Ball et al., Bioinformatics 18:395-404 (2002); and Li et al., Clinical Chemistry Journal, 48:1296-1304 (2002).

In a particular embodiment, a sample obtained from the mother is applied to a protein chip, and the proteomic pattern is generated by mass spectrometry. The pattern of the peaks within the spectrum can be analyzed by suitable bioinoformatic software, as described above.

The data presented in the Examples below provide several unique expression signatures characteristic of fetal aneuplodies. For example, as shown in Figures there are characteristic differences between the mass spectrum of normal maternal serum and maternal serum when the fetus has an aneuploidy in the molecular weight ranges of about 125 to 150 kD (area 1), about 60 to 68 kDa (area 2), about 50 to 55 kDa (area 3), about 40 to 45 kDa (area 4), about 38 to 42 kDa (area 5), about 16 to 20 kDa (area 6), and about 35 to 35 kDa (area 7). In amiotic fluid, there are characteristic expression signatures in the molecular weight regions of about 6 to 7 kDa and/or 8 to 10 kDa. Accordingly, the entire mass spectrum, or one or more of the listed regions, each representing a unique expression signature, can be used to diagnose a fetal aneuploidy using maternal serum. In addition, the mass spectrum comprising these expression signatures, or one or more of areas 1-7, in any combination, can be used as positive control in a diagnostic method for fetal aneuploidy. In addition, or alternatively, a method to diagnose an aneuploidy can include the detection of one or more proteins differentially expressed in a biological fluid of a female carrying a fetus with an aneuploidy (briefly referred to as “aneuplodal biological fluid), or fragments of such differentially expressed proteins. Differential expression includes both over- and underexpression, provided that there is a characteristic difference between the expression level of the protein in aneuploidal biological fluid relative to its expression level in normal biological fluid of the same type.

Biomarkers suitable for the detection of fetal aneuploidy using maternal serum are listed in Tables 1, 2, and 5-6. Biomarkers suitable for the detection of fetalaneuploidy using maternal amniotic fluid are listed in Table 3. Preferred biomarkers present in maternal serum and amniotic fluid, respectively, are listed in Table 4. A diagnostic assay can be based on, or can use as part of the assay, one or more of the polypeptides listed in Tables 1-6. In a specific embodiment, 1-20, or 1-15, or 1-20, or 1-15 or 1-10, or 1-9, or 1-8, or 1-7, or 1-6, or 1-5, or 1-4, or 1-3, or 1 or 2 biomarkers listed in Tables 1-6 are used, alone or combination with other biomarkers of aneuploidy, or with one or more unique expression signatures of aneuplody. Examples of potential combinations of biomarkers include the following: complement factor H and pregnancy zone protein; complement factor H and afamin; pregnancy zone protein and alpha-2-hs-glycoprotein; complement factor H, angiotensinogen, and clusterin; apolipoprotein, AMBP protein, and plasma retinol binding protein; complement factor H, afamin, angiotensinogen, and clusterin; complement factor H, afamin, pigment epithelium-derived factor, serum amyloid A protein, angiotensinogen, and clusterin; apolipoprotein E, AMBP protein, plasma retinol binding protein, serotransferrin precursor, alpha-2-macroglobulin precursor, and histidine-rich glycoprotein precursor; inter-alpha-trypsin inhibitor heavy chain H1 precursor, complement component C9 precursor, fibrinogen alpha/alpha-E chain precursor, apolipoprotein C-III precursor, leucine-rich alpha-2-glycoprotein precursor, apolipoprotein E precursor, fetuin-B precursor, and complement C4 precursor. It is noted, however, that the invention is not limited to these examples but rather all permuations of possible combinations can find use in the instant invention.

A combination of different biomarkers and/or characteristic expression signatures, as described above, might significantly improve diagnostic accuracy. For example, individual biomarkers can typically detect a fetal aneuploidy, such as Down's syndrome, in about 30% to 80% of occurrences. With a combination or biomarkers and/or characteristic expression signatures a diagnostic accurance of at least about 80%, more preferably at least about 85%, even more preferably at least about 90%, even more preferably at least about 95%, most preferably at least about 98% can be achieved. The combination of biomarkers which act independently, through distinct biological pathways is particularly advantageous, since such combinations are expected to significantly increase diagnostic sensitivity.

The diagnostic methods of the present invention are equally applicable in the first and second trimester of pregnancies essentially with the same detection rate.

While the screening methods of the invention provide an outstanding detection rate and accuracy when used alone, they can also be combined with existing screening techniques for the detection of fetal aneuploidy. Thus, the diagnostic methods herein can be combined one or more of known biomarkers, such as, for example in the case of Down's syndrome or trisomy 18, with one or more of serum biomarkers PAPP-A, α-fetoprotein (AFP), human chorionic gonadotropin (βhCG), unconjugated estriol (uE3), and inhibin A. In particular, the present screening techniques can be combined with a test using PAPP-A and βhCG as independent biomarkers, or the triple-marker serum test, based on AFP, βhCG, and uE3, especially if screening is performed in the second trimester. The test might, additionally or alternatively, include inhibin-A. Markers capable of identifying other aneuploidies that may be combined with the diagnostic methods described herein are well known in the art.

The screening assays herein can further be combined with or supplemented by other techniques in clinical or experimental use to detect fetal aneuploidy, including, ultrasonography, including transabdominal and translucent ultrasonography; various techniques to test chromosomal abnormalities; and nuchal translucency (NT) measurement.

4. Protein and Antibody Arrays

The diagnostic assays discussed above can be performed using protein arrays. In recent years, protein arrays have gained wide recognition as a powerful means to detect proteins, monitor their expression levels, and investigate protein interactions and functions. They enable high-throughput protein analysis, when large numbers of determinations can be performed simultaneously, using automated means. In the microarray or chip format, that was originally developed for DNA arrays, such determinations can be carried out with minimum use of materials while generating large amounts of data.

Although proteome analysis by 2D gel electrophoresis, 2D liquid chromotograhy, and mass spectrometry, as described above, is very effective, it does not always provide the needed high sensitivity and this might miss many proteins that are expressed at low abundance. Protein microarrays, in addition to their high efficiency, provide improved sensitivity.

Protein arrays are formed by immobilizing proteins on a solid surface, such as glass, silicon, micro-wells, nitrocellulose, PVDF membranes, and microbeads, using a variety of covalent and non-covalent attachment chemistries well known in the art. The solid support should be chemically stable before and after the coupling procedure, allow good spot morphology, display minimal nonspecific binding, should not contribute a background in detection systems, and should be compatible with different detection systems.

In general, protein microarrays use the same detection methods commonly used for the reading of DNA arrays. Similarly, the same instrumentation as used for reading DNA microarrays is applicable to protein arrays.

Thus, capture arrays (e.g. antibody arrays) can be probed with fluorescently labelled proteins from two different sources, such as normal and diseased biological fluids. In this case, the readout is based on the change in the fluorescent signal as a reflection of changes in the expression level of a target protein. Alternative readouts include, without limitation, fluorescence resonance energy transfer, surface plasmon resonance, rolling circle DNA amplification, mass spectrometry, resonance light scattering, and atomic force microscopy.

For further details, see, for example, Zhou H, et al., Trends Biotechnol. 19:S34-9 (2001); Zhu et al., Current Opin. Chem. Biol. 5:40-45-(2001); Wilson and Nock, Angew Chem Int Ed Engl 42:494-500 (2003); and Schweitzer and Kingsmore, Curr Opin Biotechnol 13:14-9 (2002). Biomolecule arrays are also disclosed in U.S. Pat. No. 6,406,921, issued Jun. 18, 2002, the entire disclosure of which is hereby expressly incorporated by reference.

Further details of the invention will be apparent from the following non-limiting examples.

### EXAMPLE I

Identification of Proteins and Polypeptides Expressed in Maternal Serum and Aminotic Fluid Samples

Materials and Methods

Maternal serum and amniotic fluid samples evaluated (matched for gestational age).

| | Control | Down's syndrome |
|---|---|---|
| 1^{st} trimester | 25 | 25 |
| 2^{nd} trimester | 25 | 25 |

Immunodepletion of Abundant Proteins in Human Serum

Human serum was depleted of six major proteins (albumin, IgG, IgA, anti-trypsin, tranferrin, and haptoglobin) using the Agilent multiple affinity system. The multiple affinity column is based on antibody-antigen interactions and optimized buffers for sample loading, washing, eluting, and regenerating. The column removes six high-abundance proteins (80-90% of total protein mass) from human serum such as albumin, IgG, IgA, anti-trypsin, transferrin, and haptoglobin, and allows the enrichment of low-abundance proteins for proteomic analysis.

Human serum (40 μl) was diluted five times with Agilent buffer A (35 μl of serum with 180 μl of buffer A). Particulates were removed by filtering through a 0.22 μm spin filter for 1 min at 16,000×g. 160 μl of the diluted serum was injected into an Agilent immunoaffinity column (4.6×100 mm) attached to a Waters HPLC system equipped with an autosampler, UV detector, and a fraction collector. The flow rate was set to 0.5 ml/min for the first 10 min with 0% B, and 10-17 min at 1 ml/min with 100% B and 17-28 min at 1 ml/min with 0% B. Low-abundance flow-through fractions 2-5 were collected, concentrated, and buffer exchanged with 10 mM Tris, pH 8.4, using 5000 MWCO filters. Protein concentration was determined using the Bio-Rad DC protein assay kit.

Fluorescent 2-DGE

High-abundance proteins from serum (1-3 mg) were depleted using Agilent immunoaffinity columns as described above. Serum proteins (20-50 μg) were then labeled with CyDye DIGE Fluor minimal dye (Amersham Biosciences) at a concentration of 100-400 pm of dye/20-50 μg of protein. Different dyes (Cy5, Cy3, and Cy2) were used to label control or test or reference serum samples. Labeled proteins were purified by acetone precipitation and dissolved in IEF buffer and rehydrated on to a 24 or 13-cm IPG strip (pH 4-7) for 12 h at room temperature. After rehydration, the IPG strip was subjected to 1-dimensional electrophoresis at 65˜70 kVhrs. The IPG strip was then equilibrated with DTT equilibration buffer I and IAA equilibration buffer II for 15 minutes sequentially, before second dimension SDS-PAGE analysis. The IPG strip was then loaded on to a 8˜16% SDS-PAGE gel and electrophoresis conducted at 80-90 V for 18 hrs to resolve proteins in the second dimension.

After the second dimension, the gel was scanned in a Typhoon 9400 scanner (Amersham) using appropriate lasers and filters with PMT voltage between 550-600 range. Images in different channels (control and test) were overlaid using selected colors, and differences were monitored using ImageQaunt software (Amersham Biosciences). Quantitation of the gel images was done using Evolution software (Nonlinear Dynamics).

For protein identification, serum proteins (500 g to 1500 μg) were subjected to 2-DGE without labeling. The gel was stained with Coomassie Blue R-250 and imaged. Individual spots were cut from the gel, destained, and digested in-gel with trypsin for 24 hrs at 37° C. The peptides were extracted with 0.1% TFA and purified using Zip Tip_{c18} pipette tips from Millipore. Western immunoblotting and immunoprecipitation

50-100 μg of serum proteins were run on 4-20% SDS-PAGE at 200 V for 60 minutes and transferred to PVDF membranes at 90 mA for 75 minutes. The membrane was blocked with 5% milk-PBST for 45 min at room temperature and incubated with 1 μg/ml primary antibody (Santa Cruz and Dako) overnight at 4° C. After washing with TBST 3 times, the membrane was incubated with an IgG-HRP secondary antibody (Sigma) for 90 min at room temperature and visualized with ECL (Pierce). For immunoprecipitation, 20 μg of primary antibody was mixed with 600 μg of serum protein and incubated at 4° C. overnight. 15 μl of protein G-Sepharose beads were then added and incubated on a shaker for 60 minutes at room temperature. The beads were washed with IP buffer for 6 times prior to elution and PAGE.

SELDI-TOF Analysis of Maternal Serum

A total of 0.5-3.0 μg protein from amniotic fluid and serum samples was spotted on a Normal-phase NP20 (SiO₂ surface), Reverse-Phase H4 (hydrophobic surface: C-16 (long-chain aliphatic), or immobilized nickel (IMAC) SELDI ProteinChip® arrays (Ciphergen Biosystems, Inc., Fremont, Calif.). After incubation at room temperature for 1 h, NP1 and H4 chips were subjected to a 5-μl water wash to remove unbound proteins and interfering substances (i.e., buffers, salts, detergents). After air-drying for 2-3 min, two 0.5-μl applications of a saturated solution of sinapinic acid in 50% acetonitrile (v/v), 0.5% trifluoroacetic acid (v/v), was added and mass analysis was performed by time-of-flight mass spectrometry in a Ciphergen Protein Biology System II (PBS II).

Isotope-Coded Affinity Tagging (ICAT)

ICAT is a recently developed complementary technique that can be used to overcome some of the limitations of 2DGE by providing protein identification and quantification data on differentially expressed proteins in control and diseased samples. The ICAT peptide labeling technique differentiates between two populations of proteins by using reactive probes that differ in isotope composition. A commercially available cleavable ICAT reagent from Applied Biosystems was used, which consists of a protein-reactive group (Iodoacetamide) that alkylates free cysteines on a protein, a ¹²C or ¹³C isotopically labeled linker region, and an affinity (biotin) tag to selectively isolate the cysteine-containing peptides. Two samples, control and diseased, are treated with the isotopically light (¹²C) or heavy (¹³C) ICAT reagents, respectively. The labeled protein mixtures are then combined, and proteolytically digested. Labeled peptides are then isolated using immobilized monomeric avidin affinity capture of the biotinylated peptides. The biotin label on the labeled peptides is then cleaved and the peptides analyzed by nanoscale liquid chromatography combined with electrospray ionization tandem mass spectrometry (LC-ESI MS/MS). The resulting MS and MS/MS spectra are analyzed using MCAT software (Waters) to determine the relative abundance of the tagged peptide pairs in control and diseased samples, and searched against a large protein sequence database to identify the protein. The control acts as an internal reference to normalize the level of protein abundance for comparative analysis. The increase or decrease in the abundance ratio provides information on up- or down-regulation.

Protein Identification

Data Acquisition and Analysis

After in-gel digestion with trypsin, samples were analyzed on a Waters hybrid quadrapole time-of-flight mass spectrometer (Q-Tof-2) connected to a Waters CapLC. The Q-Tof-2 was equipped with a regular Z-spray or nanospray source and connected to an Integrafrit or Nanoease C18 75 μm ID×15 cm×3.51 μm fused silica capillary column. The instrument was controlled by, and data were acquired on, a Compaq workstation with Windows NT and MassLynx 4.0 software. The Q-Tof-2 was calibrated using Glu1 Fibrinopeptide B by direct infusion or injection from the attached CapLC. Data-directed analysis was used. An MS/MSMS survey method was used to acquire MS/MSMS spectra. Masses of 400 to 1500 Da were scanned for MS survey, and masses of 50 to 1900 Da were scanned for MS/MS. Primary data analysis was performed on a PC with Windows 2000 and ProteinLynx Global Server v2.1 (PLGS) as well as the PEAKS de novo sequencing algorithm and our proprietary OpenSea software v1.1 (Searle et al., Analytical Chemistry 76:2220-2230 (2004)).

PLGS v2.1

Automated analysis of tandem mass spectra (MS/MS) was performed using PLGS v2.1 software (Waters). Processing parameters used either medium or slow deisotoping without any background subtraction. After processing, the deisotoped MS/MS spectra were searched against the non-redundant International Protein Index (IPI) human database (20) using a workflow with database search and automod. In the workflow, fixed modifications were carbamidomethyl C and variable modifications were oxidation M and phosphorylation STY. The automod query was run after the database search using a non-specific primary digest reagent to search for all possible modifications and substitutions.

OpenSea v1.1

The OpenSea mass-based alignment algorithm v1.1 identifies proteins from MS/MS data of peptides by aligning de novo sequences derived from the data by PEAKS to protein sequences in databases. OpenSea converts all amino acid characters into a series of masses, and these masses are compared using a dynamic programming approach.

All Q-TOF MS/MS spectra were de novo sequenced using Peaks Batch Version 2.2 (Ma et al., An effective algorithm for the peptide de novo sequencing from MS/MS spectrum, in 14th Symposium of Combinatorial Pattern Matching, 2003; Nelson et al., Analytical Chemistry 67:1153-8 (1995)) (Bioinformatics Solutions Inc., Waterloo, ON, Canada) using a mass accuracy of 0.1 AMU. Peaks reports full amino acid sequences without unknown mass regions, but assigns each amino acid in the sequence a confidence score. Sequence regions where amino acids had confidence scores below 50% were replaced by the combined mass of those amino acids. If the entire sequence had an average confidence below 50%, only amino acids that had confidence below the average confidence were combined. All sequences were analyzed with OpenSea using monoisotopic masses for calculating hypothetical parent and fragment masses and were matched with a mass accuracy of 0.25 AMU. All samples were searched against the non-redundant International Protein Index (IPI) human database.

The parameters used to identify proteins were as follows: 1) any database matches including the string “keratin” in the protein description were excluded; 2) each protein should have greater than 95% probability of occurrence by both PLGS v2.1 and OpenSea v1.1; and 3) each protein should have two or more peptides.

Mass Spectrometry-Based Immunoaffinity Assay for the Detection of Biomarkers

Protein biomarkers differentially expressed between maternal control and Down's syndrome serum identified using 2-DGE DIGE experiments are suitable for the development of a protein profile-based high-throughput screening system for the detection of fetal Down's syndrome. Individual protein biomarkers were captured from maternal serum by immunoaffinity purification and analyzed by matrix-assisted laser desorption/ionization time-of-flight mass spectrometry (MALDI-TOF MS).

Sample Preparation and Biomarker Immunoprecipitation

Serum samples were centrifuged for 15 min at 700×g to pellet blood cells. Supernatants are stored at −80° C. Each serum sample (up to 50 μL for each individual biomarker target) is diluted with binding buffer and incubated with immunoaffinity beads (Pierce; Rockford, Ill.) derivatized with 50 μg of coupled antibody. Down's syndrome target proteins were eluted from beads using a low pH, chaotropic buffer. Eluates are desalted and concentrated using ZipTip™ C4 pipette tips (Millipore; Billerica, Mass.) and spotted directly (along with sinapinic acid matrix) onto a hydrophobic/hydrophilic contrasting MALDI-TOF MS target (AnchorChip™ MTP target plate, Bruker Daltonics; Billerica, Mass.). AnchorChip targets encourage even sample distribution and crystallization, leading to higher sensitivity MALDI-MS spectra and less dependence on manual “sweet-spot” searching, making analysis more amenable to high-throughput automation.

MALDI-TOF MS Analysis

MALDI-TOF MS analysis of eluted intact protein biomarkers were performed on an Autoflex MALDI-TOF-MS mass spectrometer (Bruker Daltonics; Billerica, Mass.). The resolution specifications of the Autoflex MALDI-TOF-MS (Resolution=1000 for cytochrome c, 12361 Da, Rs=m/Δm (FWHM)) permit the detection of protein isoforms and modifications. For example, Nelson and coworkers were able to resolve isoforms of apolipoprotein E differing in mass by only 53 Da (ApoE2 and ApoE3 isoforms: 34,236.6 and 34, 183.6 Da, respectively) (228 A.T.B.n, Maternal Serum Screening. In ACOG. 1996 Washington D.C.: American College of Obstreticians and Gynecologists). The MALDI-MS was operated in linear delayed-extraction mode with positive polarity for the detection of large polypeptides and proteins (>m/z 5000). Mass spectra are acquired using an attenuated adjustable 50-Hz nitrogen laser (337 nm) with 100-200 shots per spectrum.

For adequate signal-to-noise considerations, several spectra were combined dependent on the intensity levels of the specific biomarker target of interest. Bruker MALDI-TOF mass spectrometer used has an mass accuracy in linear detection mode (used for the detection of higher mass polypeptides/proteins >m/z 5000)<100 ppm using internal calibration (for cytochrome c at m/z 12,361). External calibration is performed utilizing calibration anchors between each set of 4 sample well on Bruker MTP AnchorChip™ target plates. Post-processing analysis of acquired MALDI-MS biomarker ion signals from control and Down's syndrome samples was performed using ClinPro Tools software (Bruker Daltonics; Billerica, Mass.).

Results

A) Proteomic Profiles Using SELDI-TOF Mass Spectrometry to Detect Down's Syndrome.

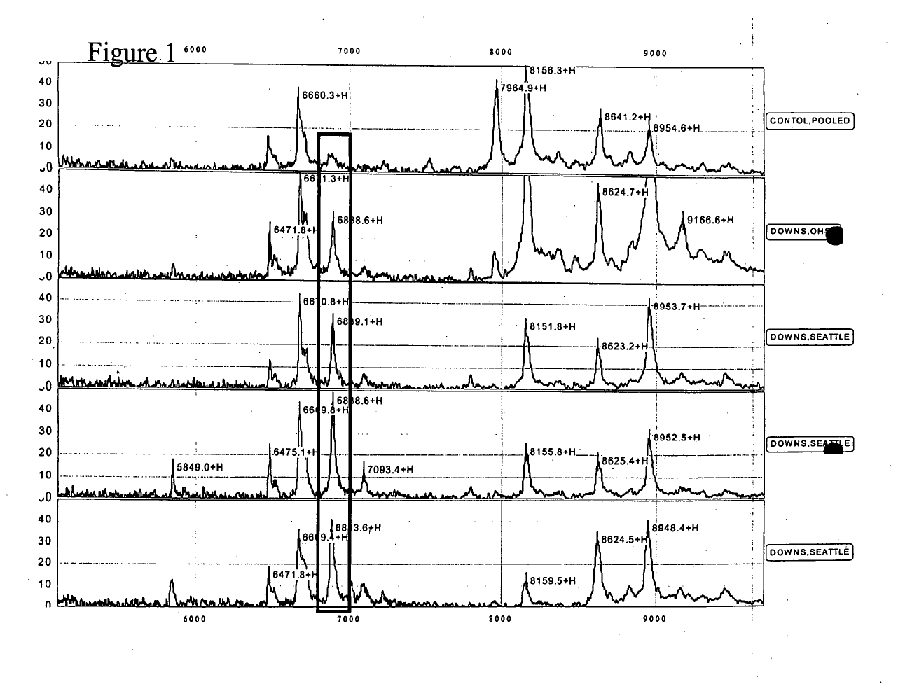

To identify the protein patterns representative of control and Down's syndrome, respectively, first low-molecular-weight proteins were enriched in serum by removing the major abundant proteins using Agilent immunoaffinity columns as described in the methods. 1-2, μg of enriched protein sample was profiled on SELDI-TOF using four different surface chemistry-enhanced capture protocols (Ciphergen Protein Chip Arrays). Data analysis using Biomarker Wizard (Ciphergen, Inc.) revealed peaks that were distinctive of control and Down's syndrome serum (FIG. 1). A subset of samples was further evaluated (Kersey et al., Proteomics 4:1985-1988 (2004)) on a MALDI-TOF (Autoflex TOF-TOF, Bruker Daltonics) and the data analyzed using Clinprot software (Bruker Daltonics). This approach also revealed a small number of distinct peaks in Down's syndrome samples. These results demonstrated that potential differences in maternal serum from Down's syndrome in the low molecular weight range can be detected by SELDI/MALDI profiling. A sensitive and specific assay utilizing these profiles unique to Down's syndrome can be developed into a proprietary high-throughput screening test.

B) Fluorescent 2-DGE.

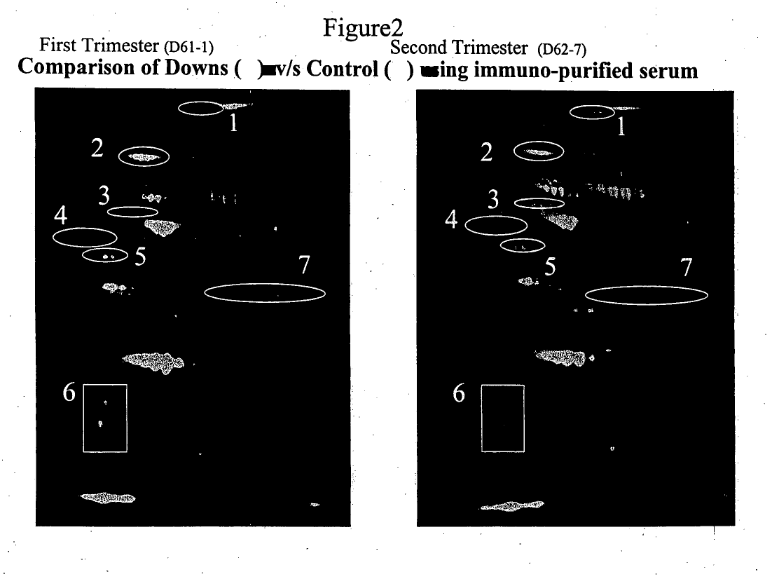

Matched pairs (control and Down's syndrome) of maternal serum samples prepared as described in the methods section were labeled with fluorescent dyes (Cy5, Cy3 and Cy2) and resolved on 2-D gels. ProteoGenix has developed proprietary high-thoughput format to screen large numbers of samples using 2-D gels and semi-quantification procedures (2-D profiles) using a fixed internal reference (pooled maternal serum) resolved on all of the gels along with control and Down's syndrome samples. As shown in FIG. 1, second-trimester maternal serum samples revealed distinct differences between control and Down's syndrome cases and significant similarity of the profiles from first and second-trimester. Quantification of intensity ratios (Phoretics software, ImageQuant software, SAS analysis) demonstrated that the significant areas of interest 1-7 (as shown in FIG. 2, high to low molecular weight) showed sensitivities ranging from about 40 to 80%. A combination of two or more areas was able to discriminate all Down's syndrome cases from controls in this matched-pair model.

To identify the potential proteins in these areas of interest, preparative 2-D gels (1-2 mg of purified protein) were used from three matched pairs of serum samples from first and second trimester. Spots from areas of interest (FIG. 2, circled areas) were punched and digested with trypsin and analyzed by LC/MS/MS (Q-TOF2). Protein identification and data analysis was performed using proprietary proteomic software (OpenSea). Each area of interest represented 2-3 proteins (Table 1). The proteins represented in the areas of interest were the same for both first and second-trimester serum samples. Proteins differentially expressed in maternal serum not represented in areas 1-7 are listed in Table 2.

Matched pairs (control and Down's syndrome) of amniotic fluid samples were analyzed as described above with fluorescent dyes (Cy5, Cy3 and Cy2) and resolved on 2-D gels. The differentially expressed proteins were identified using de novo sequencing and listed in Table 3.

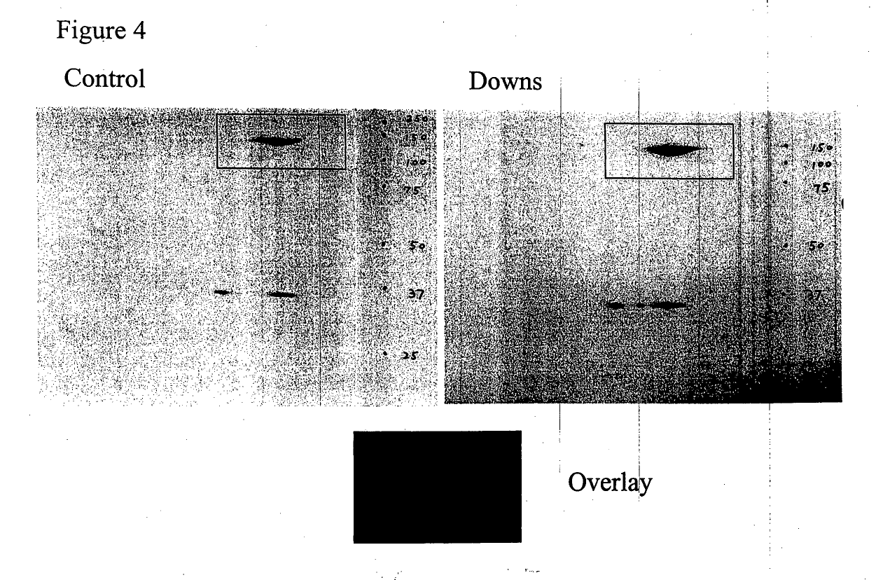

Relative quantitative differences noted in 2D fluorescent gels can be measured using Western blots. As an example antibodies to the predominant protein expressed in area 1 (Complement factor H) were used to probe a maternal serum 2D western blot resolved similarly to the 2D fluorescent gels. As shown in FIG. 4, Complement factor H was expressed at a higher level in Down's compared to control maternal serum. This demonstrates that protein biomarkers identified can be used in a standard quantification immunoassays to detect fetal Down's syndrome in maternal serum.

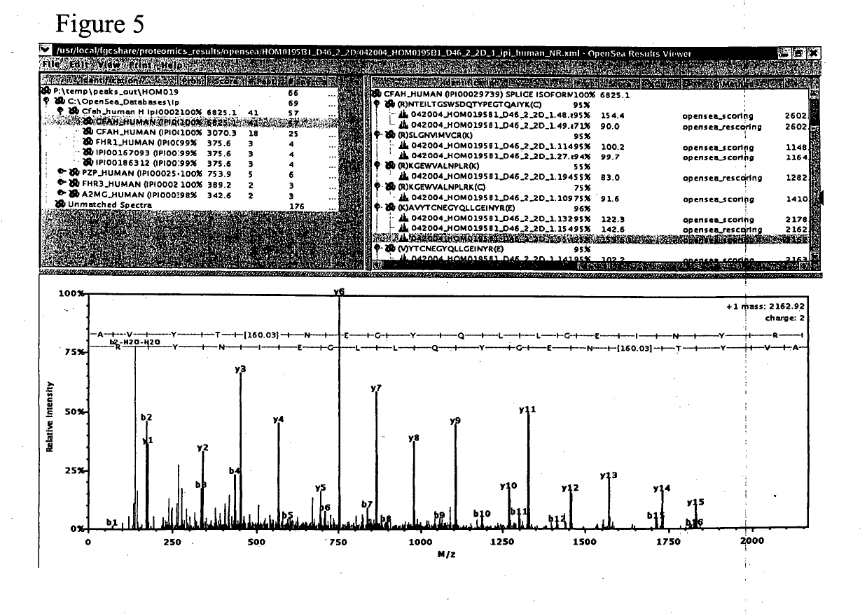

FIG. 5 is a schematic representation of de novo protein sequence identification of candidate biomarkers of Down's syndrome. In particular, the figure shows spectra representing pepide sequences that belong to Complement factor H.

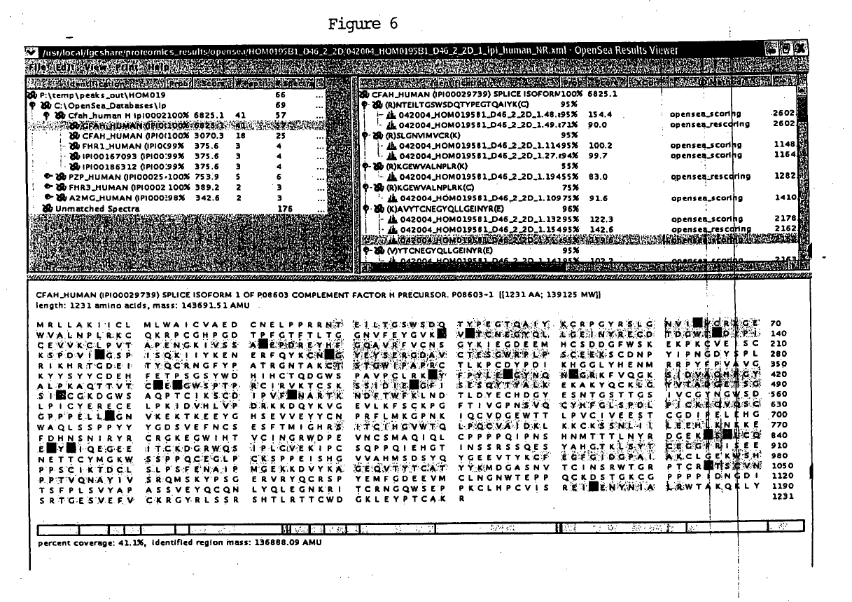

FIG. 6 is a different schematic representatino of de novo protein sequence identification of candidate biomarkers of Down's syndrome. The figure shows the sequence coverage map of peptide sequences identified that belong to Complement factor H. Lighter shading designated the peptide identified within the polypeptide sequence, and the amino acid residues marked with darker shading are potential protein modifications at the indicated positions.

Development of an Immuno-MALDI Assay to Measure Biomarkers

The fluorescent 2-D gel analysis and protein identification as presented above revealed a significant number of potential biomarkers in maternal serum in both first and second-trimester samples. An Immuno-MALDI assay platform provides an unprecedented opportunity for multianalyte analysis. Another major advantage in this assay platform is the ability to capture isoforms that are specific for a disease. It would be very difficult to develop an accurate ELISA to measure such proteolytic fragments or protein modifications. This example demonstrates the feasibility of developing a high-throughput assay employing Immuno-MALDI technology to detect Down's syndrome.

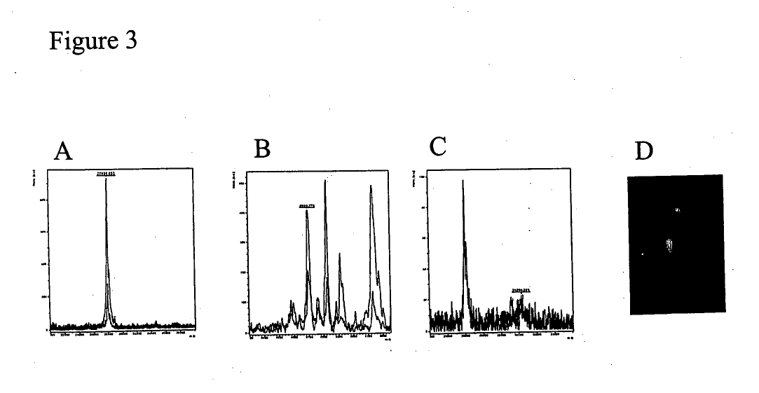

An Immuno-MALDI assay has been developed to identify the differentially expressed proteins in areas 6 and 7. Protein identification from the 2-D gel spots for this area demonstrated the presence of Apolipoproteins AI, AII, and E. Immunoprecipitation of apolipoproteins was performed using 600 μg of maternal serum samples from a matched pair of control and Down's syndrome samples. Eluents were profiled using Autoflex TOF-TOF (Bruker Daltonics) as described in the methods. As shown in FIG. 3, all three forms of apolipoprotein were detected, and apolipoprotein All showed significant quantitative differences between the two samples. Additionally, the apolipoprotein All complex also revealed distinct isoforms in Down's syndrome maternal serum.

MALDI analysis of the above sample pairs indicated down-regulation of APOA1 in Down's syndrome serum compared to control serum. When performing IP analysis on the same set of control and Down's syndrome serum using apolipoprotein A2 (APOA2) antibody, the MALDI profiles shown in FIG. 3 indicated that the relative intensity of APOA2 was again higher in the control serum versus the Down's syndrome serum (APOA2 MW=8707.9 Da). Furthermore, different species were present in control versus the Down's syndrome IPs. Thus, our data demonstrate that MALDI-TOF MS allows the evaluation of both changes in relative intensity as well as biomarker pattern changes.

This experiment demonstrates that optimization of other biomarkers identified in the 2-DGE analysis and the use of computational tools (ClinProt software) for relative quantification and optimization of statistical algorithms to develop diagnostic profiles will provide a robust high-throughput assay system. This system can be extended to distinguish other aneuploidies in the same setting through the addition of other potential targets.

Throughout the foregoing description the invention has been discussed with reference to certain embodiments, but it is not so limited. Indeed, various modifications of the invention in addition to those shown and described herein will become apparent to those skilled in the art from the foregoing description and fall within the scope of the appended claims.

### EXAMPLE II

Two-Dimensional Liquid Chromatography for the Separation and Identification of Differentially Expressed Proteins in Down's Syndrome

As a complementary strategy to 2D-DIGE analysis of proteins from maternal Control and Down's syndrome sera, a two-dimensional liquid chromatography (2D-LC) method for separating intact proteins can be employed. The 2D-LC method provides virtual 2D maps that allow for the comparison of differential protein expression between control and Down's syndrome serum samples.

Sample Preparation and 2D-LC Methodology

For comparative analysis of protein expression in maternal control and Down's syndrome sera, sets of pooled maternal sera were prepared from first trimester and from second trimester patients. All sera were immunopurified (Agilent) and buffer-exchanged for CF compatibility. Between 5-7 mg of total serum protein was pooled for each sample. The same amount of total protein was used for 2D-LC analysis for each control/Down's syndrome sample pair; first and second-trimester sample pairs were analyzed independently.

2D-LC analysis was performed on a ProteomeLab PF2D system (Beckman-Coulter; Fullerton, Calif.). Briefly, serum protein is loaded onto the first-dimension CF anion exchange column and eluted into 0.3 pH unit fractions according to protein isoelectric point (pI/pH) using a descending linear pH gradient. Each pH fraction is then separated in the second dimension by protein hydrophobicity using a nonporous C18 RP-HPLC column (48 fractions from each pH fraction). A total of 800 fractions were collected from the RP-HPLC dimension (from each sample) to be digested enzymatically with trypsin for protein identification by mass spectrometry.

MS Analysis of Collected Differential Fractions

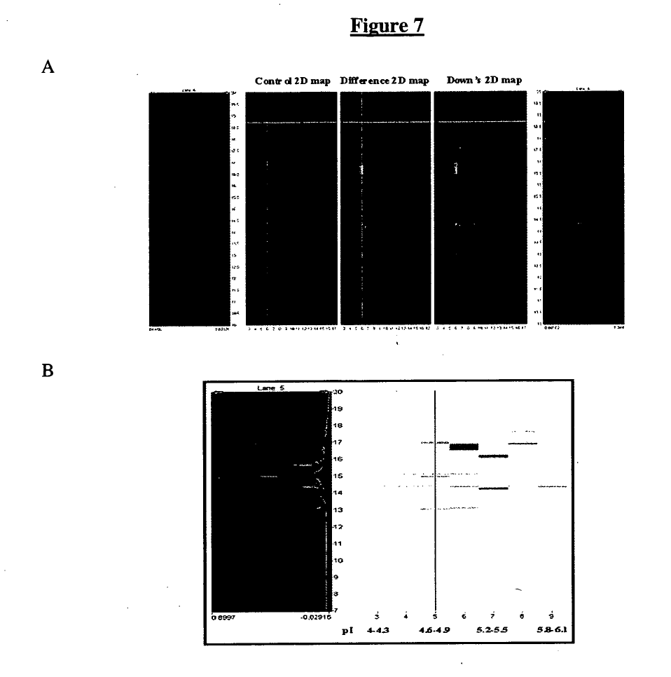

FIG. 7 shows the protein expression maps generated by the 2D-LC analysis of second trimester maternal control versus maternal Down's syndrome serum. FIG. 7A depicts the 2D-LC maps generated using ProteoVue software display the pI of the eluted protein from CF on the x-axis and the retention time, or hydrophobicity, of the eluted protein from RP-HPLC on the y-axis. FIG. 7B depicts the 2D map of the control sample is depicted in red on the left and the 2D map of the Down's syndrome sample is depicted in green on the right. The center of the figure displays the difference map (displayed separately in FIG. 7B) of the two samples, where bands seen in green are proteins up-regulated in the Down's syndrome sample and bands seen in red are proteins up-regulated in the control sample.

Bands in the difference map showing up-regulation in either Down's syndrome or control serum were digested with trypsin and prepared for protein identification analysis using an ESI-QTOF-MS/MS (QTOF2, Waters; Milford, Mass.). Using a differential intensity cutoff of at least 10-20% of the higher intensity peak from either the control or Down's syndrome sample, this corresponds to about 95 bands in the first-trimester sample set and 80 bands in the second-trimester sample set. Differential expression intensities between control and Down's syndrome fractions ranged from 0.004 AU to 0.638 AU (limit of detection for MS analysis of fraction digests is conservatively ˜0.05 AU; the AU scale for the second dimension separations reaches a maximum of ˜1.3 AU).

2D-LC Identification of Differentially Expressed Proteins in Maternal Down's Syndrome Serum

Table 5 presents a list of identified proteins showing differential peptide counts on LC/MS/MS (Q-TOF2, Waters, Inc) analysis in Down's syndrome maternal serum. (abbreviationsare T1, firstrimester; T2, second trimester maternal serum.)

### EXAMPLE III

Glycoprotein Profiles of Maternal Serum Predictive of Down's Syndrome

Glycosylation is one of the complex posttranslational modifications of proteins in eukaryotes. A systematic evaluation of the glycosylation process is a valuable tool in mining protein biomarkers, as a minor change such as a single glycosylation event can alter the fate and function of a physiologically important protein, which could be, in turn related to a particular disease or state of an organism. Changes in the glycosylation pattern or glycan structure occurring in response to cellular signals or stages of development could be used to identify diseases such as cancer. Lectin based affinity purification is the method of choice for isolating different classes of glycosylated proteins. Lectins are plant proteins, which can specifically and reversibly bind to glycan moieties in glycoproteins. The major classes and types of glycoproteins can be individually isolated from the test samples and can be used to generate a differential glycosylation profile to compare control versus disease.

Methods

Total glycoproteins, Sialic, Mannose and 0-glycosylated proteins from gestational age matched Control and DS maternal serum were purified using appropriate lectin affinity columns (Q Proteome, Quiagen).

Total glycoproteins extraction was performed using a combination of lectins, Mannose binding lectins (ConA, LCH, GNA)+Sialic acid/N-acetyl-glucosamine binding lectins (WGA, SNA). M-linked glycoproteins were extracted utilizing mannose-binding lectins (ConA, LCH, GNA). S-linked glycoproteins were extracted utilizing Sialic acid/N-acetyl-glucosamine binding lectins (WGA, SNA, MAL). ) O-linked glycoproteins were extracted utilizing Galactose/N-acetyl-galactosamine binding lectins (AIL, PNA).

Glycoproteins extracted from Control and Down's syndrome maternal serum were analyzed using 2-Dimensional fluorescent gel electrophoresis and LC/MS/MS approaches to identify potential markers for Down's syndrome. 50 ug each of the isolated Control and Down's syndrome glycoproteins were labeled with 400 pm of Cy3 and Cy5 fluorescent dyes respectively. Isoelectric focusing was performed on a pH 4-7 IPG strip on Ettan Dalt 2 IPGphor system (GE-Amresham) using appropriate voltage settings for each IPG strip length. 10-20% Tris-Glycine gels were used for the second dimension PAGE. Differential fluorescent image for each gel was acquired using Typhoon Variable mode imager (GE-Amersham) using excitation wavelengths for Cy3 and Cy5. Differentially expressed proteins spots were visualized using ImageQuant (GE-Amersham) software, excised from the gel, and digested with trypsin for protein identification on a mass spectrometer (Q-TOF 2, Waters Inc).

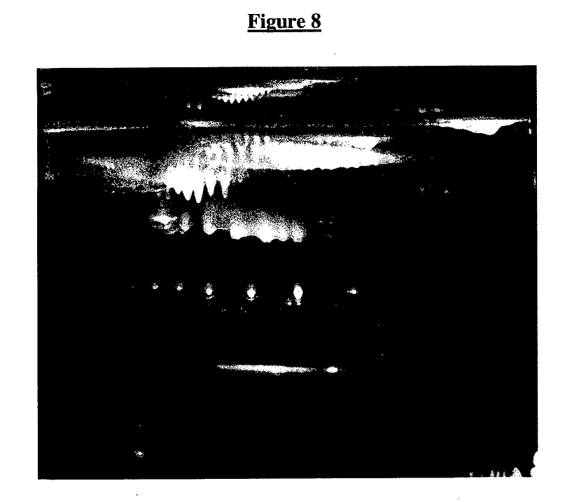

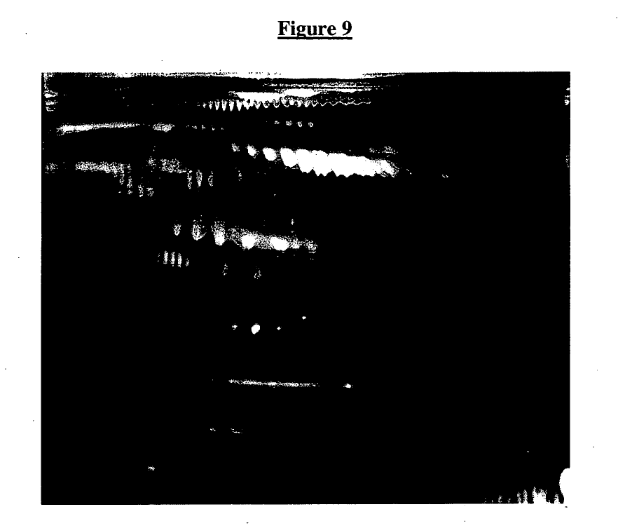

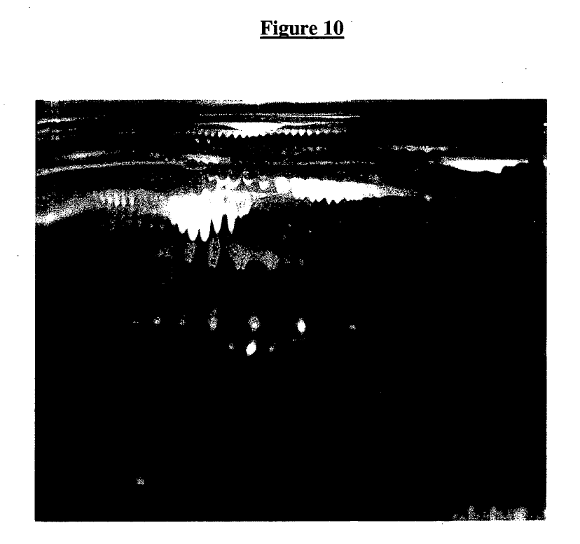

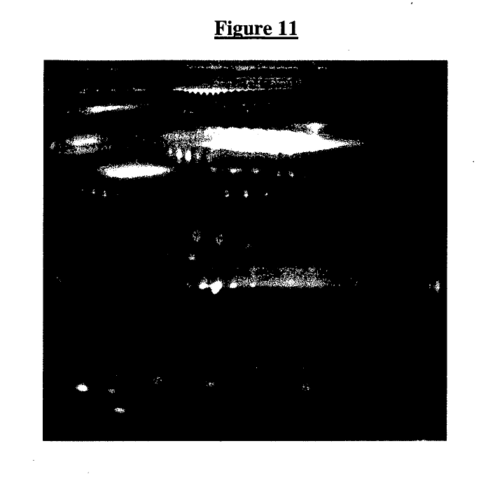

FIGS. 8-11 represent unique differential expression profiles of glycoproteins in maternal serum in Down's syndrome.

Areas showing differences, red or green were punched from the gels, digested with trypsin and protein identification was confirmed using LC/MS/MS.

Total glycoprotein mixtures extracted from 1st and 2nd trimester control and Down's syndrome maternal serum samples were digested with trypsin and analyzed using LC/MS/MS. Glycoproteins representing differences (greater number of total peptides for each protein) were compiled and compared with the glycoproteins identified from differentially expressed spots from 2-dimensional gels and the list of glycoproteins identified in Down's syndrome maternal serum is presented in Table 6.

All references cited throughout the description, and the references cited therein, are hereby expressly incorporated by reference in their entirety.

| | Differential expression of proteins in Human Maternal Serum | | | | |
|---|---|---|---|---|---|
| | in areas 1-7as determined by 2D-DIGE | | | | |
| | SwissProt | | | Max peptides | |
| | Accession | | | in one | Max |
| Area | Number | Protein ID | Description | sample | coverage |
| 1 | P08603 | CFAH_HUMAN | COMPLEMENT FACTOR H | 36 | 41% |
| 1 | P20742 | PZP_HUMAN | PREGNANCY ZONE PROTEIN | 7 | 10% |
| 1 | Q02985 | FHR3_HUMAN | COMPLEMENT FACTOR H-RELATED PROTEIN 3 | 2 | 10% |
| 2 | P43652 | AFAM_HUMAN | AFAMIN | 13 | 28% |
| 2 | Q14624 | ITH4_HUMAN | INTER-ALPHA-TRYPSIN INHIBITOR HEAVY CHAIN H4 | 7 | 13% |
| 2 | P01019 | ANGT_HUMAN | ANGIOTENSINOGEN | 6 | 17% |
| 3 | P01019 | ANGT_HUMAN | ANGIOTENSINOGEN | 12 | 35% |
| 3 | P02774 | VTDB_HUMAN | VITAMIN D-BINDING PROTEIN | 12 | 50% |
| 3 | P01008 | ANT3_HUMAN | ANTITHROMBIN-III | 3 | 33% |
| 4 | P02765 | A2HS_HUMAN | ALPHA-2-HS-GLYCOPROTEIN | 9 | 38% |
| 4 | P01019 | ANGT_HUMAN | ANGIOTENSINOGEN | 7 | 21% |
| 4 | P04004 | VTNC_HUMAN | VITRONECTIN | 3 | 8% |
| 5 | P02647 | APA1_HUMAN | APOLIPOPROTEIN A-I | 11 | 58% |
| 5 | P10909 | CLUS_HUMAN | CLUSTERIN | 6 | 26% |
| 5 | P01024 | CO3_HUMAN | COMPLEMENT C3 | 9 | 8% |
| 6 | P02647 | APA1_HUMAN | APOLIPOPROTEIN A I | 5 | 25% |
| 6 | P06727 | APA4_HUMAN | APOLIPOPROTEIN A IV | 4 | 14% |
| 7 | P02649 | APE_HUMAN | APOLIPOPROTEIN E | 11 | 47% |
| 7 | O75636 | FCN3_HUMAN | FICOLIN 3 | 5 | 28% |
| 7 | P01028 | CO4_HUMAN | COMPLEMENT C4 | 3 | 2% |

TABLE 2
Differentially expressed proteins in Human Control & Downs Serum

| SwissProt | IPI | | |
|---|---|---|---|
| Accession | Accession | | |
| Number | Number | Protein ID | Proteins identified |
| P02763 | | A1AG_HUMAN | Alpha 1 acid glycoprotein |
| P04217 | | A1BG_HUMAN | Alpha 1B Glycoprotein |
| P02760 | | AMBP_HUMAN | AMBP protein |
| P01024 | | CO3_HUMAN | Anaphylotoxin C3A |
| P02647 | | APA1_HUMAN | Apolipoprotein A-1 |
| P02652 | | APA2_HUMAN | Apolipoprotein A-II |
| P02654 | | APC1_HUMAN | Apolipoprotein C-I |
| P02655 | | APC2_HUMAN | Apolipoprotein C-II |
| P02749 | | APOH_HUMAN | Beta-2 glycoprotein |
| P05109 | | S108_HUMAN | Calgranulin A |
| P00450 | | CERU_HUMAN | Ceruloplasmin |
| P01028 | | CO4_HUMAN | Complement C4 |
| P01024 | | CO3_HUMAN | Complement C-III |
| P08603 | | CFAH_HUMAN | Complement Factor H (splice isofo [symbol] |
| P02679 | | FIBG_HUMAN | Fibrinogen-gamma chain |
| P00737 | | HPT1_HUMAN | Haptoglobin 1 |
| P00738 | | HPT_HUMAN | Haptoglobin 2 |
| P00739 | | HPTR_HUMAN | Haptoglobin related protein |
| P02790 | | HEMO_HUMAN | Hemopexin |
| P36955 | | PEDF_HUMAN | Pigment Epithelium-Derived Facto [symbol] |
| P05155 | | IC1_HUMAN | Plasma Protease C1 Inhibitor |
| P02775 | | SZ07_HUMAN | Platelet basic protein |
| P02735 | | SAA_HUMAN | Serum amyloid A protein |
| | IPI00257664 | | Similar to Ceruloplasmin |
| | IPI00053956 | | Similar to Dead H ASP GLU AL |
| P04004 | | VTNC_HUMAN | Vitronectin |
| P25311 | | ZA2G_HUMAN | Zinc alpha 2 glycoprotein |

TABLE 3
Differentially expressed proteins
in Human Control & Downs Amniotic Fluid

| SwissProt | IPI | | |
|---|---|---|---|
| Accession | Accession | | |
| Number | Number | Protein ID | Description |
| P02765 | | A2HS_HUMAN | Alpha 2 HS Glycoprotein |
| P02760 | | AMBP_HUMAN | AMBP protein |
| P02647 | | APA1_HUMAN | Apolipoprotein A-1 |
| P01884 | | B2MG_HUMAN | Beta-2-microglobulin |
| | IPI00178276 | | BPOZ splice variant |
| P02452 | | CA11_HUMAN | Collagen alpha 1 (I) chain |
| P02461 | | CA13_HUMAN | Collagen alpha 1 (III) chain |
| P01034 | | CYTC_HUMAN | Cystatin C |
| | IPI00073904 | | D 10S 102 |
| | IPI00010341 | EMBP_HUMAN | Eosinophil Granule Major |
| | | | Basi |
| P09466 | | PAEP_HUMAN | Glycodelin (GD) |
| | | | (Pregnancy |
| | | | associated protein) |
| | IPI00334832 | | Hypothetical 177AA 20495 |
| | IPI00182398 | | Hypothetical protein |
| | | | FLJ40785 |
| | IPI00246890 | | Hypothetical protein |
| | | | XP_299919 |
| | IPI00178229 | | LAMRL5 |
| P51884 | | LUM_HUMAN | Lumican |
| | IPI00178198 | | Nuclear factor I-A |
| P02753 | | RETB_HUMAN | Plasma retinol binding |
| | | | protein |
| | IPI00306589 | | Ubiquitin B 229 AA 25762 |

TABLE 4

| SwissProt | | |
|---|---|---|
| Accession | | |
| Number | Protein ID | Description |
| P08603 | CFAH_HUMAN | complement factor H |
| P20741 | PZP_HUMAN | pregnancy zone protein |
| P43652 | AFAM_HUMAN | afamin |
| P01019 | ANGT_HUMAN | angiotensinogen |
| P02765 | A2HS_HUMAN | alpha-2-hs-glycoprotein |
| P10909 | CLUS_HUMAN | clusterin |
| P02647 | APA1_HUMAN | apolipoprotein AI |
| P06727 | APA4-HUMAN | apolipoprotein AIV |
| P02649 | APE_HUMAN | apolipoprotein E |
| P36933 | PEDF_HUMAN | pigment epithelium-derived |
| | | factor |
| P02735 | SAA_HUMAN | serum amyloid A protein |
| P02760 | AMBP_HUMAN | AMBP protein |
| P02753 | RETB_HUMAN | plasma retinol binding |
| | | protein |

TABLE 5

| | | Tri- |
|---|---|---|
| Protein | Description | mester |
| A1AG | Alpha-1-acid glycoprotein 1 precursor | T1 |
| A1AH | Alpha-1-acid glycoprotein 2 precursor | T1 |
| A1BG | Alpha-1B-glycoprotein precursor | T1, T2 |
| A2GL | Leucine-rich alpha-2-glycoprotein precursor | T2 |
| A2HS | Alpha-2-HS-glycoprotein precursor | T1, T2 |
| A2MG | Alpha-2-macroglobulin precursor | T1 |
| AFAM | Afamin precursor | T2 |
| ANT3 | Antithrombin-III precursor | T1, T2 |
| APA1 | Apolipoprotein A-I precursor | T1, T2 |
| APA2 | Apolipoprotein A-II precursor | T2 |
| APA4 | Apolipoprotein A-IV precursor | T1, T2 |
| APC1 | Apolipoprotein C-I precursor | T2 |
| APC2 | Apolipoprotein C-II Precursor | T2 |
| APC3 | Apolipoprotein C-III precursor | T1, T2 |
| APOD | Apolipoprotein D precursor | T1 |
| APOE | Apolipoprotein E precursor | T1 |
| CERU | Ceruloplasmin precursor | T1, T2 |
| CFAB | Complement factor B precursor | T1 |
| CFAH | Complement factor H precursor | T1, T2 |
| CFAI | Complement factor I precursor | T1 |
| CLUS | Clusterin precursor | T1, T2 |
| CO3 | Complement C3 precursor | T1, T2 |
| CO4 | Complement C4 precursor | T2 |
| CO6 | Complement component C6 precursor | T1, T2 |
| CO7 | Complement component C7 precursor | T1, T2 |
| F13B | Coagulation factor XIII B chain precursor | T1, T2 |
| FA12 | Coagulation factor XII precursor | T2 |
| HEMO | Hemopexin precursor | T1, T2 |
| HRG | Histidine-rich glycoprotein precursor | T1, T2 |
| ITH4 | Inter-alpha-trypsin inhibitor heavy chain H4 precursor | T1, T2 |
| KNG | Kininogen precursor | T1, T2 |
| PLMN | Plasminogen precursor | T1 |
| PSG1 | Pregnancy-specific beta-1-glycoprotein 1 precursor | T2 |
| RETB | Plasma retinol-binding protein precursor | T2 |
| SHBG | Sex hormone-binding globulin precursor | T2 |
| TETN | Tetranectin precursor | T1, T2 |
| THRB | Prothrombin precursor | T2 |
| TTHY | Transthyretin precursor | T1, T2 |
| VTDB | Vitamin D-binding protein precursor | T1, T2 |
| ZA2G | Zinc-alpha-2-glycoprotein precursor | T1, T2 |

TABLE 6

| | | UniprotKB/ | |
|---|---|---|---|
| | | Swiss- | |
| | | Prot/TrEMBL | |
| | IPI Accession | Accession | |
| Protein ID | Number | Number | Description |
| TRFE_HUMAN | IPI00022463 | P02787 | SEROTRANSFERRIN PRECURSOR. P02787 [[698 AA; 77050 MW]] |
| A1AT_HUMAN | IPI00305457 | Q9P173 | ALPHA-1-ANTITRYPSIN PRECURSOR. P01009 [[418 AA; 46737 MW]] |
| A2MG_HUMAN | IPI00032256 | Q59F47 | ALPHA-2-MACROGLOBULIN PRECURSOR. P01023 [[1474 AA; 163278 MW]] |
| CO3_HUMAN | IPI00164623 | P01024 | COMPLEMENT C3 PRECURSOR [Contains: |
| | | | C3A ANAPHYLATOXIN]. P01024 [[1664 AA; 187235 MW]] |
| ANGT_HUMAN | IPI00032220 | P01019 | ANGIOTENSINOGEN PRECURSOR [Contains: |
| | | | ANGIOTENSIN I (ANG I) ANGIOTENSIN II (ANG II) ANGIOTENSIN III |
| | | | (ANG III) (DES-ASP[1]-ANGIOTENSIN II)]. P01019 [[485 AA; 53154 MW]] |
| CERU_HUMAN | IPI00017601 | P00450 | CERULOPLASMIN PRECURSOR. P00450 [[1065 AA; 122205 MW]] |
| HPT_HUMAN | IPI00019571 | P00738 | HAPTOGLOBIN PRECURSOR. P00738 [[416 AA; 46271 MW]] |
| ANT3_HUMAN | IPI00032179 | P01008 | ANTITHROMBIN-III PRECURSOR. P01008 [[464 AA; 52602 MW]] |
| HEMO_HUMAN | IPI00022488 | P02790 | HEMOPEXIN PRECURSOR. P02790 [[462 AA; 51676 MW]] |
| A1AG_HUMAN | IPI00022429 | P02763 | ALPHA-1-ACID GLYCOPROTEIN 1 PRECURSOR. |
| | | | P02763 [[201 AA; 23512 MW]] |
| APA1_HUMAN | IPI00021841 | P02647 | APOLIPOPROTEIN A-I PRECURSOR. P02647 [[267 AA; 30778 MW]] |
| IPI00216722 | IPI00216722 | P04217 | ALPHA 1B-GLYCOPROTEIN. [[495 AA; 54254 MW]] |
| KNG_HUMAN | IPI00215894 | P01042-2 | SPLICE ISOFORM LMW OF P01042 KININOGEN PRECURSOR |
| | | | (ALPHA-2-THIOL PROTEINASE INHIBITOR) [Contains: BRADYKININ]. |
| | | | P01042-2 [[427 AA; 47883 MW]] |
| ITH2_HUMAN | IPI00305461 | P19823 | INTER-ALPHA-TRYPSIN INHIBITOR HEAVY CHAIN H2 |
| | | | PRECURSOR. P19823 [[947 AA; 106596 MW]] |
| A2HS_HUMAN | IPI00022431 | P02765 | ALPHA-2-HS-GLYCOPROTEIN PRECURSOR. |
| | | | P02765 [[367 AA; 39325 MW]] |
| AACT_HUMAN | IPI00032215 | P01011-2 | ALPHA-1-ANTICHYMOTRYPSIN, PRECURSOR. P01011 |
| | | | [[433 AA; 48637 MW]] |
| ITH4_HUMAN | IPI00218192 | Q14624-2 | SPLICE ISOFORM 2 OF Q14624 INTER-ALPHA-TRYPSIN |
| | | | INHIBITOR HEAVY CHAIN H4 PRECURSOR (ITI HEAVY CHAIN H4) |
| | | | (INTER-ALPHA-INHIBITOR HEAVY CHAIN 4) (INTER-ALPHA- |
| | | | TRYPSIN INHIBITOR FAMILY HEAVY CHAIN-RELATED PROTEIN) |
| | | | (IHRP) (PLASMA KALLIKREIN SENSITIVE GLYCOPROTEIN 120) |
| | | | (PK-120) (GP120) (PRO1851) [Contains: GP57]. |
| | | | Q14624-2 [[914 AA; 101242 MW]] |
| CFAH_HUMAN | IPI00029739 | P08603-1 | SPLICE ISOFORM 1 OF P08603 COMPLEMENT FACTOR H |
| | | | PRECURSOR. P08603-1 [[1231 AA; 139125 MW]] |
| IC1_HUMAN | IPI00291866 | P05155 | PLASMA PROTEASE C1 INHIBITOR PRECURSOR. |
| | | | P05155 [[500 AA; 55154 MW]] |
| IPI00154742 | IPI00154742 | Q8N355 | HYPOTHETICAL PROTEIN. [[237 AA; 24897 MW]] |
| HEP2_HUMAN | IPI00292950 | P05546 | HEPARIN COFACTOR II PRECURSOR. P05546 [[499 AA; 57071 MW]] |
| CFAB_HUMAN | IPI00019591 | P00751-1 | SPLICE ISOFORM 1 OF P00751 COMPLEMENT FACTOR B |
| | | | PRECURSOR. P00751-1 [[764 AA; 85533 MW]] |
| ZA2G_HUMAN | IPI00166729 | P25311 | ALPHA-2-GLYCOPROTEIN 1, ZINC. P25311 [[298 AA; 34259 MW]] |
| VTNC_HUMAN | IPI00298971 | P04004 | VITRONECTIN PRECURSOR (SERUM SPREADING FACTOR) |
| | | | (S-PROTEIN) (V75) [Contains: VITRONECTIN V65 SUBUNIT |
| | | | VITRONECTIN V10 SUBUNIT SOMATOMEDIN B]. |
| | | | P04004 [[478 AA; 54306 MW]] |
| IPI00061246 | IPI00061246 | Q96E61 | HYPOTHETICAL PROTEIN. [[236 AA; 24713 MW]] |
| ITH1_HUMAN | IPI00292530 | P19827 | INTER-ALPHA-TRYPSIN INHIBITOR HEAVY CHAIN H1 |
| | | | PRECURSOR. P19827 [[911 AA; 101389 MW]] |
| CO9_HUMAN | IPI00022395 | P02748 | COMPLEMENT COMPONENT C9 PRECURSOR. |
| | | | P02748 [[559 AA; 63173 MW]] |
| FIBA_HUMAN | IPI00021885 | P02671-1 | SPLICE ISOFORM ALPHA-E OF P02671 FIBRINOGEN |
| | | | ALPHA/ALPHA-E CHAIN PRECURSOR [Contains: |
| | | | FIBRINOPEPTIDE A]. P02671-1 [[866 AA; 94973 MW]] |
| FIBB_HUMAN | IPI00298497 | P02675 | FIBRINOGEN BETA CHAIN |
| | | | PRECURSOR [Contains: FIBRINOPEPTIDE B]. |
| | | | P02675 [[491 AA; 55928 MW]] |
| FIBG_HUMAN | IPI00021891 | P02679-1 | SPLICE ISOFORM GAMMA-B OF P02679 FIBRINOGEN |
| | | | GAMMA CHAIN PRECURSOR. P02679-1 [[453 AA; 51512 MW]] |
| THRB_HUMAN | IPI00019568 | P00734 | PROTHROMBIN PRECURSOR. P00734 [[622 AA; 70037 MW]] |
| CLUS_HUMAN | IPI00291262 | P10909 | CLUSTERIN PRECURSOR. P10909 [[476 AA; 55192 MW]] |
| A1BG_HUMAN | IPI00022895 | P04217 | ALPHA-1B-GLYCOPROTEIN PRECURSOR. |
| | | | P04217 [[495 AA; 54209 MW]] |
| A1AH_HUMAN | IPI00020091 | P19652 | ALPHA-1-ACID GLYCOPROTEIN 2 PRECURSOR. |
| | | | P19652 [[201 AA; 23603 MW]] |
| APOD_HUMAN | IPI00006662 | P05090 | APOLIPOPROTEIN D PRECURSOR. |
| | | | P05090 [[189 AA; 21276 MW]] |
| PZP_HUMAN | IPI00025426 | P20742 | PREGNANCY ZONE PROTEIN PRECURSOR. |
| | | | P20742 [[1482 AA; 163836 MW]] |
| HRG_HUMAN | IPI00022371 | P04196 | HISTIDINE-RICH GLYCOPROTEIN PRECURSOR. |
| | | | P04196 [[525 AA; 59578 MW]] |
| IPI00166866 | IPI00166866 | Q8N5K4 | HYPOTHETICAL PROTEIN. [[499 AA; 53376 MW]] |
| SHBG_HUMAN | IPI00023019 | P04278-1 | SPLICE ISOFORM 1 OF P04278 SEX HORMONE-BINDING |
| | | | GLOBULIN PRECURSOR. P04278-1 [[402 AA; 43779 MW]] |
| PLMN_HUMAN | IPI00019580 | P00747 | PLASMINOGEN PRECURSOR (EC 3.4.21.7) [Contains: ANGIOSTATIN]. |
| | | | P00747 [[810 AA; 90569 MW]] |
| APC3_HUMAN | IPI00021857 | P02656 | APOLIPOPROTEIN C-III PRECURSOR. |
| | | | P02656 [[99 AA; 10852 MW]] |
| A2GL_HUMAN | IPI00022417 | P02750 | LEUCINE-RICH ALPHA-2-GLYCOPROTEIN PRECURSOR. |
| | | | P02750 [[347 AA; 38178 MW]] |
| APE_HUMAN | IPI00021842 | P02649 | APOLIPOPROTEIN E PRECURSOR. P02649 [[317 AA; 36154 MW]] |
| FETB_HUMAN | IPI00005439 | Q9UGM5 | FETUIN-B PRECURSOR. Q9UGM5 [[382 AA; 42094 MW]] |
| IPI00007884 | IPI00007884 | Q9UL83 | MYOSIN-REACTIVE IMMUNOGLOBULIN LIGHT CHAIN VARIABLE |
| | | | REGION. [[108 AA; 11834 MW]] |
| C1S_HUMAN | IPI00017696 | P09871 | COMPLEMENT C1S COMPONENT PRECURSOR. |
| | | | P09871 [[688 AA; 76684 MW]] |
| AMBP_HUMAN | IPI00022426 | P02760 | AMBP PROTEIN PRECURSOR [Contains: ALPHA-1-MICROGLOBULIN |
| | | | (PROTEIN HC) (COMPLEX-FORMING GLYCOPROTEIN |
| | | | HETEROGENEOUS IN CHARGE) (ALPHA-1 MICROGLYCOPROTEIN) |
| | | | INTER-ALPHA-TRYPSIN INHIBITOR LIGHT CHAIN (ITI-LC) |
| | | | (BIKUNIN) (HI-30)]. P02760 [[352 AA; 38999 MW]] |
| CO4_HUMAN | IPI00032258 | P01028 | COMPLEMENT C4 PRECURSOR [Contains: |
| | | | C4A ANAPHYLATOXIN]. P01028 [[1744 AA; 192771 MW]] |

What is claimed is:

1. A method for diagnosis of fetal aneuploidy, comprising comparing the proteomic profile of a test sample of a maternal biological fluid with a normal or a reference proteomic profile of the same type of biological fluid, and determining the presence of fetal aneuploidy if the proteomic profile of said test sample shows at least one unique expression signature representing at least one biomarker selected from the group consisting of the biomarkers listed in Tables 1-2 and 5-6, absent from said normal proteomic profile or present in said reference proteomic profile.

2. The method of claim 1 wherein said test sample is obtained from a pregnant female human.

3. The method of claim 1 wherein said proteomic profile is a mass spectrum.

4. The method of claim 1 wherein test sample is maternal serum.

5. The method of claim 4 wherein said unique expression signature is in one or more of molecular weight regions 16 to 20 kDa, 35 to 38 kDa, 38 to 42 kDa, 40 to 45 kDa, 50 to 55 kDa, 60 to 68 kDa, and 125 to 150 kDa.

6. The method of claim 2 which is performed in the first trimester of pregnancy.

7. The method of claim 2 which is performed in the second trimester of pregnancy.

8. The method of claim 1 further comprising determining in said test sample the level of transcribed mRNA or the level of translated protein of at least one additional biomarker of fetal aneuploidy, and confirming the presence of fetal aneuploidy if said level of transcribed mRNA or level of translated protein is different relative to its level in a normal biological sample.

9. The method of claim 8 wherein said fetal aneuploidy is Down's syndrome, trisomy 13, trisomy 18, X chromosome trisomy, X chromosome monosomy, Kleinfelter's syndrome (XXY genotype), or XYY syndrome (XYY genotype).

10. The method of claim 1 wherein said fetal aneuploidy is Down's syndrome, trisomy 13, trisomy 18, X chromosome trisomy, X chromosome monosomy, Kleinfelter's syndrome (XXY genotype), or XYY syndrome (XYY genotype).

11. The method of claim 8 wherein said additional biomarker is selected from the group consisting of PAPP-A, a-fetoprotein (AFP), human chorionic gonadotropin (bhCG), unconjugated estriol (uE3), and inhibin A.

12. The method of claim 11 wherein the level of transcribed mRNA or the level of translated PAPP-A and bhCG are determined.

13. The method of claim 12 wherein the level of transcribed mRNA or the level of translated AFP, bhCG, and uE3 are additionally determined.

14. The method of claim 13 wherein the level of transcribed mRNA or the level of translated inhibin-A is additionally determined.

15. The method of claim 2 further comprising subjecting the pregnant female human to one or more of additional diagnostic techniques.

16. The method of claim 15 wherein said additional diagnostic techniques are selected from the group consisting of ultrasonography, techniques to test chromosomal abnormalities, and nuchal translucency (NT) measurement.

17. The method of claim 1 comprising comparison of the unique expression signature of more than one of said biomarkers.

18. The method of claim 1 wherein said biomarker or biomarkers are selected from the group consisting of complement factor H (CFAH_HUMAN, SwissProt Accession No. P08603); pregnancy zone protein (PZP_HUMAN; SwissProt Accession No. P20741); afamin (AFAM_HUMAN; SwissProt Accession No. P43652); angiotensinogen (ANGT_HUMAN; SwissProt Accession No. P01019); alpha-2-hs-glycoprotein (A2HS_HUMAN; SwissProt Accession No. P02765); clusterin (CLUS_HUMAN; SwissProt Accession No. P10909); apolipoprotein AI (APA1_HUMAN; SwissProt Accession No. P02647); apolipoprotein AIV (APA4_HUMAN; SwissProt Accession No. P06727); apolipoprotein E (APE_HUMAN; SwissProt Accession No. P02649); pigment epithelium-derived factor (PEDF_HUMAN; SwissProt Accession No. P36955); serum amyloid A protein (SAA_HUMAN; SwissProt Accession No. P02735); AMBP protein (AMBP_HUMAN; SwissProt Accession No. P02760); plasma retinol binding protein (RETB_HUMAN; SwissProt Accession No. P02753); serotransferrin precursor (TRFE_HUMAN; SwissProt Accession No. P02787); alpha-1-antitrypsin precursor (A1AT_HUMAN; SwissProt Accession No. P01009); alpha-2-macroglobulin precursor (A2MG_HUMAN; SwissProt Accession No. P01023); complement C3 precursor (CO3_HUMAN; SwissProt Accession No. P01024); angiotensinogen precursor (ANGT_HUMAN; SwissProt Accession No. P01019); ceruloplasmin precursor (CERU_HUMAN; SwissProt Accession No. P00450); haptoglobin precursor (HPT_HUMAN; SwissProt Accession No. P00738); antithrombin-III precursor (ANT3_HUMAN; SwissProt Accession No. P01008); hemopexin precursor (HEMO_HUMAN; SwissProt Accession No. P02790); alpha-1-acid glycoprotein 1 precursor (A1AG_HUMAN; SwissProt Accession No. P02763); apolipoprotein A-I precursor (APA1_HUMAN; SwissProt Accession No. P02647); alpha 1b-glycoprotein (SwissProt Accession No. P04217); kininogen precursor (KNG_HUMAN; SwissProt Accession No. P01042-2); inter-alpha-trypsin inhibitor heavy chain H2 precursor (ITH2_HUMAN; SwissProt Accession No. P19823); alpha-2-hs-glycoprotein precursor (A2HS_HUMAN; SwissProt Accession No. P02765); alpha-1-antichymotrypsin precursor (AACT_HUMAN; SwissProt Accession No. P01011); inter-alpha-trypsin inhibitor heavy chain H4 precursor (ITH4_HUMAN; SwissProt Accession No. Q14624-2); complement factor H precursor (CFAH_HUMAN; SwissProt Accession No. P08603-1); plasma protease C1 inhibitor precursor (IC1_HUMAN; SwissProt Accession No. P05155); heparin cofactor II precursor (HEP2_HUMAN SwissProt Accession No. P05546); complement factor B precursor (CFAB_HUMAN; SwissProt Accession No. P00751-1); alpha-2-glycoprotein 1, zinc (ZA2G_HUMAN; SwissProt Accession No. P25311); vitronectin precursor (VTNC_HUMAN SwissProt Accession No. P04004); inter-alpha-trypsin inhibitor heavy chain H1 precursor (ITH1_HUMAN; SwissProt Accession No. P19827); complement component C9 precursor (CO9_HUMAN; SwissProt Accession No. P02748); fibrinogen alpha/alpha-E chain precursor (FIBA_HUMAN; SwissProt Accession No. P02671-1); fibrinogen beta chain precursor (FIBB_HUMAN; SwissProt Accession No. P02675); fibrinogen gamma chain precursor (FIBG_HUMAN; SwissProt Accession No. P02679-1); prothrombin precursor (THRB_HUMAN; SwissProt Accession No. P00734); clusterin precursor (CLUS_HUMAN; SwissProt Accession No. P10909); alpha-1B-glycoprotein precursor (A1BG_HUMAN; SwissProt Accession No. P04217); alpha-1-acid glycoprotein 2 precursor (A1AH_HUMAN; SwissProt Accession No. P19652); apolipoprotein D precursor (APOD_HUMAN; SwissProt Accession No. P05090); pregnancy zone protein precursor (PZP_HUMAN; SwissProt Accession No. P20742); histidine-rich glycoprotein precursor (HRG_HUMAN; SwissProt Accession No. P04196); sex hormone-binding globulin precursor (SHBG_HUMAN; SwissProt Accession No. P04278-1); plasminogen precursor (PLMN_HUMAN; SwissProt Accession No. P00747); apolipoprotein C-III precursor (APC3_HUMAN; SwissProt Accession No. P02656); leucine-rich alpha-2-glycoprotein precursor (A2GL_HUMAN; SwissProt Accession No. P02750); apolipoprotein E precursor (APE_HUMAN; SwissProt Accession No. P02649); fetuin-B precursor (FETB_HUMAN; SwissProt Accession No. Q9UGM5); myosin-reactive immunoglobulin light chain variable region (SwissProt Accession No. Q9UL83); complement C1S component precursor (C1S_HUMAN; SwissProt Accession No. P09871); ambp protein precursor (AMBP_HUMAN; SwissProt Accession No. P02760); and complement C4 precursor (CO4_HUMAN; SwissProt Accession No. P01028).

19. The method of claim 18 comprising comparison of the unique expression signature of more than one of said biomarkers.

20. The method of claim 1 wherein said biomarkers are complement factor H (CFAH_HUMAN, SwissProt Accession No. P08603); and pregnancy zone protein (PZP_HUMAN; SwissProt Accession No. P20741).

21. The method of claim 1 wherein said biomarkers are complement factor H (CFAH_HUMAN, SwissProt Accession No. P08603); and afamin (AFAM_HUMAN; SwissProt Accession No. P43652).

22. The method of claim 1 wherein said biomarkers are pregnancy zone protein (PZP_HUMAN; SwissProt Accession No. P20741); and alpha-2-hs-glycoprotein (A2HS_HUMAN; SwissProt Accession No. P02765).

23. The method of claim 1 wherein said biomarkers are complement factor H (CFAH_HUMAN, SwissProt Accession No. P08603); angiotensinogen (ANGT_HUMAN; SwissProt Accession No. P01019); and clusterin (CLUS_HUMAN; SwissProt Accession No. P10909).

24. The method of claim 1 wherein said biomarkers are apolipoprotein E (APE_HUMAN; SwissProt Accession No. P02649); AMBP protein (AMBP_HUMAN; SwissProt Accession No. P02760); and plasma retinol binding protein (RETB_HUMAN; SwissProt Accession No. P02753).

25. The method of claim 1 wherein said biomarkers are complement factor H (CFAH_HUMAN, SwissProt Accession No. P08603); afamin (AFAM_HUMAN; SwissProt Accession No. P43652); angiotensinogen (ANGT_HUMAN; SwissProt Accession No. P01019); and clusterin (CLUS_HUMAN; SwissProt Accession No. P10909).

26. The method of claim 1 wherein said biomarkers are complement factor H (CFAH_HUMAN, SwissProt Accession No. P08603); afamin (AFAM_HUMAN; SwissProt Accession No. P43652); pigment epithelium-derived factor (PEDF_HUMAN; SwissProt Accession No. P36955); serum amyloid A protein (SAA_HUMAN; SwissProt Accession No. P02735); angiotensinogen (ANGT_HUMAN; SwissProt Accession No. P01019); and clusterin (CLUS_HUMAN; SwissProt Accession No. P10909).

27. The method of claim 1 wherein said biomarkers are apolipoprotein E (APE_HUMAN; SwissProt Accession No. P02649); AMBP protein (AMBP_HUMAN; SwissProt Accession No. P02760); plasma retinol binding protein (RETB_HUMAN; SwissProt Accession No. P02753); serotransferrin precursor (TRFE_HUMAN; SwissProt Accession No. P02787); alpha-2-macroglobulin precursor (A2MG_HUMAN; SwissProt Accession No. P01023); and histidine-rich glycoprotein precursor (HRG_HUMAN; SwissProt Accession No. P04196).

28. The method of claim 1 wherein said biomarkers are inter-alpha-trypsin inhibitor heavy chain H1 precursor (ITH1_HUMAN; SwissProt Accession No. P19827); complement component C9 precursor (CO9_HUMAN; SwissProt Accession No. P02748); fibrinogen alpha/alpha-E chain precursor (FIBA_HUMAN; SwissProt Accession No. P02671-1); apolipoprotein C-III precursor (APC3_HUMAN; SwissProt Accession No. P02656); leucine-rich alpha-2-glycoprotein precursor (A2GL_HUMAN; SwissProt Accession No. P02750); apolipoprotein E precursor (APE_HUMAN; SwissProt Accession No. P02649); fetuin-B precursor (FETB_HUMAN; SwissProt Accession No. Q9UGM5); and complement C4 precursor (CO4_HUMAN; SwissProt Accession No. P01028).

29. The method of claim 1 wherein said proteomic profiles include at least one glycoprotein.

30. The method of claim 29 wherein said at least one glycoprotein is selected from the group consisting of sialic acid glycoproteins, mannose binding glycoproteins, and O-linked glycoproteins.

31. The method of claim 1 wherein said fetal aneuploidy is an autosomal aneuploidy.

32. The method of claim 31 wherein said autosomal aneuploidy is a trisomy of chromosomes 13, 18, or 21.

33. The method of claim 1 wherein said fetal aneuploidy is a sex chromosome aneuploidy.

34. The method of claim 33 wherein said sex chromosome aneuploidy is selected from the group consisting of: X chromosome trisomy, X chromosome monosomy, Kleinfelter's syndrome (XXY genotype), and XYY syndrome (XYY genotype).

35. A method for diagnosis of fetal aneuploidy, comprising comparing the proteomic profile of a test sample of a maternal biological fluid with a normal or a reference proteomic profile of the same type of biological fluid, and determining the presence of fetal aneuploidy if the proteomic profile of said test sample shows at least one unique expression signature representing at least one biomarker selected from the group consisting of the biomarkers listed in Table 3, absent from said normal proteomic profile or present in said reference proteomic profile.

36. The method of claim 35 wherein said test sample is obtained from a pregnant female human.

37. The method of claim 35 wherein said proteomic profile is a mass spectrum.

38. The method of claim 35 wherein the test sample is maternal amniotic fluid.

39. The method of claim 38 wherein said unique expression signature is in one or both of molecular weight regions of 6 to 7 kDa and 8 to 10 kDa.

40. The method of claim 36 which is performed in the first trimester of pregnancy.

41. The method of claim 36 which is performed in the second trimester of pregnancy.

42. The method of claim 35 further comprising determining in said test sample the level of transcribed mRNA or the level of translated protein of at least one additional biomarker of fetal aneuploidy, and confirming the presence of fetal aneuploidy if said level of transcribed mRNA or level of translated protein is different relative to its level in a normal biological sample.

43. The method of claim 2 wherein said fetal aneuploidy is Down's syndrome, trisomy 13, trisomy 18, X chromosome trisomy, X chromosome monosomy, Kleinfelter's syndrome (XXY genotype), or XYY syndrome (XYY genotype).

44. The method of any one of claim 42 wherein said fetal aneuploidy is Down's syndrome, trisomy 13, trisomy 18, X chromosome trisomy, X chromosome monosomy, Kleinfelter's syndrome (XXY genotype), or XYY syndrome (XYY genotype).

45. The method of claim 42 wherein said additional biomarker is selected from the group consisting of PAPP-A, a-fetoprotein (AFP), human chorionic gonadotropin (bhCG), unconjugated estriol (uE3), and inhibin A.

46. The method of claim 45 wherein the level of transcribed mRNA or the level of translated PAPP-A and bhCG are determined.

47. The method of claim 46 wherein the level of transcribed mRNA or the level of translated AFP, bhCG, and uE3 are additionally determined.

48. The method of claim 47 wherein the level of transcribed mRNA or the level of translated inhibin-A is additionally determined.

49. The method of claim 36 further comprising subjecting the pregnant female human to one or more of additional diagnostic techniques.

50. The method of claim 49 wherein said additional diagnostic techniques are selected from the group consisting of ultrasonography, techniques to test chromosomal abnormalities, and nuchal translucency (NT) measurement.

51. The method of claim 35 comprising comparison of the unique expression signature of more than one of said biomarkers.

52. The method of claim 35 wherein said biomarker or biomarkers are selected from the group consisting of complement factor H (CFAH_HUMAN, SwissProt Accession No. P08603); pregnancy zone protein (PZP_HUMAN; SwissProt Accession No. P20741); afamin (AFAM_HUMAN; SwissProt Accession No. P43652); angiotensinogen (ANGT_HUMAN; SwissProt Accession No. P01019); alpha-2-hs-glycoprotein (A2HS_HUMAN; SwissProt Accession No. P02765); clusterin (CLUS_HUMAN; SwissProt Accession No. P10909); apolipoprotein AI (APA1_HUMAN; SwissProt Accession No. P02647); apolipoprotein AIV (APA4_HUMAN; SwissProt Accession No. P06727); apolipoprotein E (APE_HUMAN; SwissProt Accession No. P02649); pigment epithelium-derived factor (PEDF_HUMAN; SwissProt Accession No. P36955); serum amyloid A protein (SAA_HUMAN; SwissProt Accession No. P02735); AMBP protein (AMBP_HUMAN; SwissProt Accession No. P02760); plasma retinol binding protein (RETB_HUMAN; SwissProt Accession No. P02753); serotransferrin precursor (TRFE_HUMAN; SwissProt Accession No. P02787); alpha-1-antitrypsin precursor (A1AT_HUMAN; SwissProt Accession No. P01009); alpha-2-macroglobulin precursor (A2MG_HUMAN; SwissProt Accession No. P01023); complement C3 precursor (CO3_HUMAN; SwissProt Accession No. P01024); angiotensinogen precursor (ANGT_HUMAN; SwissProt Accession No. P01019); ceruloplasmin precursor (CERU_HUMAN; SwissProt Accession No. P00450); haptoglobin precursor (HPT_HUMAN; SwissProt Accession No. P00738); antithrombin-III precursor (ANT3_HUMAN; SwissProt Accession No. P01008); hemopexin precursor (HEMO_HUMAN; SwissProt Accession No. P02790); alpha-1-acid glycoprotein 1 precursor (A1AG_HUMAN; SwissProt Accession No. P02763); apolipoprotein A-I precursor (APA1_HUMAN; SwissProt Accession No. P02647); alpha 1b-glycoprotein (SwissProt Accession No. P04217); kininogen precursor (KNG_HUMAN; SwissProt Accession No. P01042-2); inter-alpha-trypsin inhibitor heavy chain H2 precursor (ITH2_HUMAN; SwissProt Accession No. P19823); alpha-2-hs-glycoprotein precursor (A2HS_HUMAN; SwissProt Accession No. P02765); alpha-1-antichymotrypsin precursor (AACT_HUMAN; SwissProt Accession No. P01011); inter-alpha-trypsin inhibitor heavy chain H4 precursor (ITH4_HUMAN; SwissProt Accession No. Q14624-2); complement factor H precursor (CFAH_HUMAN; SwissProt Accession No. P08603-1); plasma protease C1 inhibitor precursor (IC1_HUMAN; SwissProt Accession No. P05155); heparin cofactor II precursor (HEP2_HUMAN SwissProt Accession No. P05546); complement factor B precursor (CFAB_HUMAN; SwissProt Accession No. P00751-1); alpha-2-glycoprotein 1, zinc (ZA2G_HUMAN; SwissProt Accession No. P25311); vitronectin precursor (VTNC_HUMAN SwissProt Accession No. P04004); inter-alpha-trypsin inhibitor heavy chain H1 precursor (ITH1_HUMAN; SwissProt Accession No. P19827); complement component C9 precursor (CO9_HUMAN; SwissProt Accession No. P02748); fibrinogen alpha/alpha-E chain precursor (FIBA_HUMAN; SwissProt Accession No. P02671-1); fibrinogen beta chain precursor (FIBB_HUMAN; SwissProt Accession No. P02675); fibrinogen gamma chain precursor (FIBG_HUMAN; SwissProt Accession No. P02679-1); prothrombin precursor (THRB_HUMAN; SwissProt Accession No. P00734); clusterin precursor (CLUS_HUMAN; SwissProt Accession No. P10909); alpha-1B-glycoprotein precursor (A1BG_HUMAN; SwissProt Accession No. P04217); alpha-1-acid glycoprotein 2 precursor (A1AH_HUMAN; SwissProt Accession No. P19652); apolipoprotein D precursor (APOD_HUMAN; SwissProt Accession No. P05090); pregnancy zone protein precursor (PZP_HUMAN; SwissProt Accession No. P20742); histidine-rich glycoprotein precursor (HRG_HUMAN; SwissProt Accession No. P04196); sex hormone-binding globulin precursor (SHBG_HUMAN; SwissProt Accession No. P04278-1); plasminogen precursor (PLMN_HUMAN; SwissProt Accession No. P00747); apolipoprotein C-III precursor (APC3_HUMAN; SwissProt Accession No. P02656); leucine-rich alpha-2-glycoprotein precursor (A2GL_HUMAN; SwissProt Accession No. P02750); apolipoprotein E precursor (APE_HUMAN; SwissProt Accession No. P02649); fetuin-B precursor (FETB_HUMAN; SwissProt Accession No. Q9UGM5); myosin-reactive immunoglobulin light chain variable region (SwissProt Accession No. Q9UL83); complement C1S component precursor (C1S_HUMAN; SwissProt Accession No. P09871); ambp protein precursor (AMBP_HUMAN; SwissProt Accession No. P02760); and complement C4 precursor (CO4_HUMAN; SwissProt Accession No. P01028).

53. The method of claim 52 comprising comparison of the unique expression signature of more than one of said biomarkers.

54. The method of claim 35 wherein said biomarkers are complement factor H (CFAH_HUMAN, SwissProt Accession No. P08603); and pregnancy zone protein (PZP_HUMAN; SwissProt Accession No. P20741).

55. The method of claim 35 wherein said biomarkers are complement factor H (CFAH_HUMAN, SwissProt Accession No. P08603); and afamin (AFAM_HUMAN; SwissProt Accession No. P43652).

56. The method of claim 2 wherein said biomarkers are pregnancy zone protein (PZP_HUMAN; SwissProt Accession No. P20741); and alpha-2-hs-glycoprotein (A2HS_HUMAN; SwissProt Accession No. P02765).

57. The method of claim 2 wherein said biomarkers are complement factor H (CFAH_HUMAN, SwissProt Accession No. P08603); angiotensinogen (ANGT_HUMAN; SwissProt Accession No. P01019); and clusterin (CLUS_HUMAN; SwissProt Accession No. P 10909).

58. The method of claim 2 wherein said biomarkers are apolipoprotein E (APE_HUMAN; SwissProt Accession No. P02649); AMBP protein (AMBP_HUMAN; SwissProt Accession No. P02760); and plasma retinol binding protein (RETB_HUMAN; SwissProt Accession No. P02753).

59. The method of claim 2 wherein said biomarkers are complement factor H (CFAH_HUMAN, SwissProt Accession No. P08603); afamin (AFAM_HUMAN; SwissProt Accession No. P43652); angiotensinogen (ANGT_HUMAN; SwissProt Accession No. P01019); and clusterin (CLUS_HUMAN; SwissProt Accession No. P10909).

60. The method of claim 2 wherein said biomarkers are complement factor H (CFAH_HUMAN, SwissProt Accession No. P08603); afamin (AFAM_HUMAN; SwissProt Accession No. P43652); pigment epithelium-derived factor (PEDF_HUMAN; SwissProt Accession No. P36955); serum amyloid A protein (SAA_HUMAN; SwissProt Accession No. P02735); angiotensinogen (ANGT_HUMAN; SwissProt Accession No. P01019); and clusterin (CLUS_HUMAN; SwissProt Accession No. P10909).

61. The method of claim 2 wherein said biomarkers are apolipoprotein E (APE_HUMAN; SwissProt Accession No. P02649); AMBP protein (AMBP_HUMAN; SwissProt Accession No. P02760); plasma retinol binding protein (RETB_HUMAN; SwissProt Accession No. P02753); serotransferrin precursor (TRFE_HUMAN; SwissProt Accession No. P02787); alpha-2-macroglobulin precursor (A2MG_HUMAN; SwissProt Accession No. P01023); and histidine-rich glycoprotein precursor (HRG_HUMAN; SwissProt Accession No. P04196).

62. The method of claim 2 wherein said biomarkers are inter-alpha-trypsin inhibitor heavy chain H1 precursor (ITH1_HUMAN; SwissProt Accession No. P19827); complement component C9 precursor (CO9_HUMAN; SwissProt Accession No. P02748); fibrinogen alpha/alpha-E chain precursor (FIBA_HUMAN; SwissProt Accession No. P02671-1); apolipoprotein C-III precursor (APC3_HUMAN; SwissProt Accession No. P02656); leucine-rich alpha-2-glycoprotein precursor (A2GL_HUMAN; SwissProt Accession No. P02750); apolipoprotein E precursor (APE_HUMAN; SwissProt Accession No. P02649); fetuin-B precursor (FETB_HUMAN; SwissProt Accession No. Q9UGM5); and complement C4 precursor (CO4_HUMAN; SwissProt Accession No. P01028).

63. The method of claim 2 wherein said proteomic profiles include at least one glycoprotein.

64. The method of claim 63 wherein said at least one glycoprotein is selected from the group consisting of sialic acid glycoproteins, mannose binding glycoproteins, and O-linked glycoproteins.

65. The method of claim 2 wherein said fetal aneuploidy is an autosomal aneuploidy.

66. The method of claim 65 wherein said autosomal aneuploidy is a trisomy of chromosomes 13, 18, or 21.

67. The method of claim 2 wherein said fetal aneuploidy is a sex chromosome aneuploidy.

68. The method of claim 67 wherein said sex chromosome aneuploidy is selected from the group consisting of: X chromosome trisomy, X chromosome monosomy, Kleinfelter's syndrome (XXY genotype), and XYY syndrome (XYY genotype).
